(19) United States
(12) Patent Application Publication
(10) Pub. No.: US 2017/0360332 A1
(43) Pub. Date: Dec. 21, 2017

(54) GAIT MECHANICS FOR USE IN FACILITATING ENHANCED MUSCLE CONTRACTILE CAPABILITIES

(71) Applicant: Muscle Activation, Englewood, CO (US)

(72) Inventor: Greg Roskopf, Englewood, CO (US)

(21) Appl. No.: 15/623,167

(22) Filed: Jun. 14, 2017

Related U.S. Application Data
(60) Provisional application No. 62/350,575, filed on Jun. 15, 2016.

(51) Int. Cl. A61B 5/11; A61H 99/00; A61B 5/00; G09B 19/00

(52) U.S. Cl. CPC A61B 5/112; G09B 19/003; A61H 99/00; A61B 5/0053; A61B 5/4519; A61H 2201/1253

(57) ABSTRACT

Methods, systems and kits are disclosed for facilitating a subject's gait and gait mechanics. Movement patterns with muscle hierarchy are also disclosed.

### RELATED APPLICATIONS

This application claims priority under 35 U.S.C. 119 (e) to U.S. Provisional Patent Application Ser. No. 62/350,575, entitled “GAIT MECHANICS FOR USE IN FACILITATING ENHANCED MUSCLE CONTRACTILE CAPABILITIES”, filed Jun. 15, 2016, and is related to U.S. patent application Ser. No. 15/065,665, entitled “METHODS, SYSTEMS AND KITS FOR ENHANCED MUSCLE CONTRACTILE CAPABILITIES”, filed Mar. 9, 2016, the disclosure of which is hereby incorporated by reference in its entirety.

### TECHNICAL FIELD

The disclosure generally relates to methods, systems and kits for maintaining and improving the gait and gait mechanism of a subject.

### BACKGROUND OF THE INVENTION

Conventional muscle treatment is based on the diagnoses and treatment of muscle pain and weakness in hopes of limiting pain and improving an individual's ability to exercise and physically perform. Where pain is the issue, pain medications and anti-inflammatories are prescribed and in some cases injected into a problematic area. Conventional techniques also attempt to directly lengthen or change a muscle via stretching, heating, kneading and/or foam rolling the target muscle. These techniques are dictated by identification and treatment on the specific muscle, or muscle location of the pain or weakness.

Techniques have also been developed on the principle that human movement and exercise is fundamental to health and that loss of muscle contractile efficiency may be demonstrated as a loss of motion and a decrease in physical performance. This is particularly true during aging, where our movement patterns tend to deteriorate and become more dysfunctional. Identification and treatment of muscle pain and weakness is a persistent problem, particularly when related to the aging process, in need of additional solutions.

The present disclosure and embodiments described herein are directed toward providing novel solutions to improving and maintaining an individual's muscle contractile abilities.

The present invention is directed toward overcoming one or more of the problems discussed above.

### BRIEF DESCRIPTION OF THE DRAWINGS

The described embodiments will be readily understood by the following detailed description in conjunction with the accompanying drawings.

FIG. 1 shows an illustration of raising the set-point via a strain/tissue deformation versus stress/stimulus graph.

FIG. 2 shows a flow diagram consistent with one embodiment described herein.

### SUMMARY OF THE INVENTION

Embodiments herein include the identification and characterization of 43 primary movement patterns (herein “pattern(s)”) that account for movement in the human body. The classification of the 43 patterns is based on each pattern's function. Each pattern includes a primary muscle and one or more secondary muscles. The 43 movement patterns provide a bilateral hierarchy within and between each pattern, which allows for a pre-determined and sequential treatment schedule for any one muscle in a subject.

Use of the pre-determined and sequential schedule is combined with application of a muscle specific stress, testing, and treatment to the muscle. All embodiments herein include application of at least a muscle specific stress. As discussed in greater detail below, a muscle specific stress (MSAS, for example) is required to lock-in a target muscle for subsequent treatment. Addition of a general stress to the muscle specific stress compounds the stress relationship, and draws out the locked-in state of the muscles being stressed, tested and treated. However, a general stress is only useful in combination with a muscle specific stress. As such, all embodiments herein are based on the 43 movement patterns, and the application of at least a muscle specific stress, and where deemed appropriate, a general stress. Where both a general stress and muscle specific stress are applied together within the 43 movement patters, the combination is called a compound stress.

Embodiments herein provide for the application of a compound stress to a subject, within the identified bilateral hierarchy, so as to activate and lock-in a target muscle in the subject for testing and, if necessary, treatment. General stressors include, for example, various exercise regiments based on a subject's gait. Muscle Specific Stressors (MSAS) are established for each of the 43 muscle pattern's based on a muscle pattern's primary function (see below). As such, and in one embodiment, a subject participates in a gait based exercise regiment, followed by a muscle pattern hierarchy determined MSAS, where the muscle that was stressed by MSAS undergoes further testing and, if necessary, treatment, in accordance with the description herein.

Through the application of compound stressors, and the testing, and treatment based on the muscle pattern hierarchy, embodiments herein provide methods and systems for raising the tolerance and/or stability of one or more muscles in a subject. In some embodiments, the methods and systems are designed to enhance the contractile efficiency of some or all of a subject's muscles. In still other embodiments, the methods and systems herein are designed to tie a subject's 43 muscle groups into a subject's central pattern generator (CPG) so as to better orchestrate the subject's rhythmic movement patterns, for example the subject's gait.

### DESCRIPTION

Muscle specific stress application, using the methods, systems and kits described herein, initially cause a target muscle to exceed its current set-point level, thereby creating inhibition in that target muscle. Once inhibition has been achieved, the target muscle is ready for transition to an activated state, also referred to herein as the muscle being ‘locked in.’

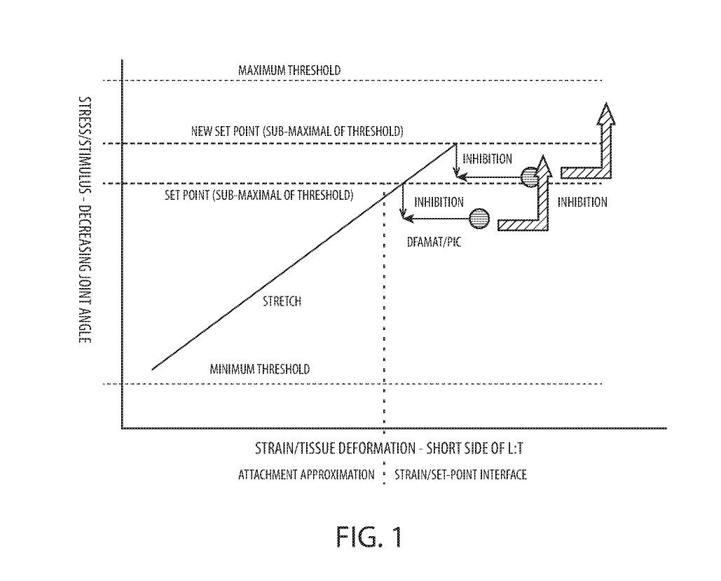

A specific stress can be re-applied to the target muscle after the muscle has been activated, which will respond in the absence or with a lower state of inhibition to the applied stress. This process of stressing (and then treating) a target muscle allows for an increase in the target muscle's set-point towards the target muscles current maximum tolerance and stability level. The establishment of a new set point for the target muscle ultimately widens the physiological operating window of the target muscle. These methods, systems and kits described herein may be repeated over a period of time to incrementally increase a target muscle's set point (see FIG. 1). As referred to herein, a widening of the physiological operating window of a target muscle means that the muscle has increase contractile efficiency and has an improved ability to tolerate greater amounts of force. As a target muscle is specifically stressed and treated, that target muscle enters a “locked-in state” where the cycle of treatment becomes more effective than for a similarly treated muscle not in a “locked in” state.

A process of facilitating the “locking in” state for a muscle (or group of muscles) can be achieved by application of a general stress, for example requiring a subject to move at an escalating gait over the course of a predetermined amount of time, prior to application of the specific stress. The combination of a general stress and a muscle specific stress is referred to herein as compounding the stress or simply as a compound stress. Where the compound stress is focused on the subject's gait pattern, the general stress is not only facilitating the “locked in” state for a muscle, but it is also providing an overall benefit for the subject's rhythmic-based mobility (not found when a specific stressor is applied in the absence of the general stressor). Note that a general stress does not “lock-in” a target muscle, rather a general stress draws-out the locked-in pattern. As such, a general stress is always provided or accompanied by a specific stress, but not vice versa. A general stress is applied and combined with a specific stress at the health practitioners discretion. Only upon adding a general stress to a specific stress does the stress become a compound stress.

In this embodiment, therefore, the subject undergoes a compound stress, is tested and then treated, as described more fully below, to facilitate tying the subject's muscle groups into the subject's central pattern generator, which orchestrates the rhythmic movement patterns necessary for the subject's gait. In various aspects, the general stress can be applied to the subject prior to the testing and treatment of each bilateral muscle pattern, for example, general stress, specific stress, testing, treatment on muscle group 1, general stress, specific stress, testing, treatment on muscle group 2, general stress, specific stress, testing, treatment on muscle group 3, and so on.

### Methods For Activating a Target Muscle:

Generally, methods disclosed herein provide for the application of stress, and thereby treatment, in a pre-determined priority based on the hierarchy of muscles between each pattern, i.e., a muscle's macro-pattern. In addition, this hierarchy of muscles extends within the patterns themselves thereby establishing a micro-pattern wherein each muscle within a pattern has a hierarchy including one primary muscle and one or more secondary muscles. The patterns are ordered bilaterally from left to right (left first, then right).

Embodiments herein provide that each of the identified 43 movement patterns have one primary muscle (a subject has 43 primary muscles) and a corresponding number of secondary muscles. As such, the hierarchy between a subject's muscles described herein can be described between muscle patterns, between primary muscles, as well as within a pattern itself (one primary and a number of secondary muscles). A macro-pattern is the overall hierarchy between all 43 movement patterns, and a micro-pattern is the hierarchy between muscles within any one muscle pattern. All patterns are bilateral.

The hierarchy of muscles within the human body provides an unexpected window, from and during which, one or more of a subject's muscles, within a pattern, is more effectively treated to enhance that muscle groups set point. The hierarchy can also be utilized to methodically enhance the set point of individual patterns including some or all of a subject's muscles and thereby facilitate the subject's musculature in general or facilitate maintenance of a subject's musculature against aging and health defects (this is particularly true, where a compound stress is applied to the subject so as to improve the subject's rhythmic movement patterns necessary for the subject's gait, a typical problem of aging).

As such, each macro- and micro-pattern has been mapped herein to identify the order within which each muscle, after being stressed, is first tested and then, after weakness is identified, treated. In this manner each muscle within the body, targeted or not, can be treated in a way to maximize the effectiveness of the treatment. Within the compound stress, it is the muscle specific stress that is hierarchy specific, where the general stress is applied in a similar manner for all muscles (as is described further below).

In some embodiments the hierarchy established and mapped in the present disclosure is between two or more patterns, i.e., between any two or more primary muscles. In one embodiment, the hierarchy provides a muscle specific stress and treatment hierarchy for any two, three, four, five, six, seven, . . . forty one, forty two, forty three primary muscles in the absence of a pattern's secondary muscles. As such, priority is mapped to start the process at a first primary muscle and extend through the macro-pattern of all the primary muscles (43). Testing and treatment of a subject's macro-pattern then includes bilaterally challenging each pattern via muscle specific stress application (left first, then right), a prioritized primary muscle to identify a bilateral weakness. When a primary muscle requires treatment (see below) the treatment is applied. Regardless, once bilateral testing and treatment (if necessary) is concluded on the first or most prioritized primary muscle, the health care professional moves onto the next highest prioritized muscle group, testing for bilateral weakness. This process holds true for the hierarchy of the 43 primary muscles (see Table 1). In some embodiments all 43 primary muscles are tested and treated in the order as disclosed in Table 1, from 1-43. In other embodiments, a primary muscle is tested and treated in an order where the primary muscle having a lower group number is always tested and treated prior to a next primary muscle, for example from 1, 7, to 9. In this way a health care professional may wish to treat a primary muscle of group 22 and deem it appropriate to start the process at the primary muscle of group 3, then 4-9, then 15-22, for example. At no time would the process start with a priority number higher than the target group primary number, e.g., 27, 25 then 22, for example. However, a health care professional may deem it necessary or advantageous to continue treatment beyond the target number, for example continue on from 22 to treat 27-31 and 40-43 (in order). In some instances where the subject has been treated via the methods described herein or is an elite athlete, fewer primary muscles may need to be tested and treated. It is also envisioned that the health care professional start the process and move bilaterally from group 1 to group 22 (in this example).

In another embodiment, priority is mapped to start the bilateral process at a first muscle in the first muscle group, i.e., the group's left primary muscle, and extend through that group's secondary muscle hierarchy, the micro-pattern. In some embodiments, the process is then continued to the first muscle, primary muscle, in the next prioritized pattern. Embodiments herein include establishing the hierarchy between two or more of the 43 patterns, three or more of the 43 total patterns, four or more of the 43 total patterns (4/43) and so on (5/43), (6/43), (7/43), (8/43), (9/43), (10/43) . . . (42/43), (43/43). In this way, the hierarchy has been identified for and between all 43 patterns (primary and secondary muscles) providing a pattern that establishes enhanced treatment for all muscles within all 43 patterns.

In some embodiments, the application of a stress is used in conjunction with muscle weakness testing (AMC&S) and treatment to effectively treat all muscles within all 43 patterns in the disclosed herein hierarchy. However, any combination can be achieved as long as the processes herein follows the hierarchy established and described, i.e., started with a pattern's primary muscle and, where appropriate, that pattern's secondary muscles, in a pattern prioritized above the next to be treated pattern's primary, and where appropriate, secondary muscles. As noted for the macro-pattern, the micro-patterns are established bilaterally. So, the hierarchy begins with the left primary muscle and moves to the pattern's right primary muscle, then to the left highest secondary muscle and then right highest secondary muscle, and so on.

As such, a first muscle (left, primary) in a pattern having the highest priority of the 43 patterns (referred to herein as pattern 1) has been identified all the way through to the last muscle (a right, secondary muscle) in the lowest priority pattern (referred to herein as muscle pattern 43). Typically and unexpectedly, maximum benefit is achieved for any one muscle group when compound stressor application and testing is applied to the associated muscles within the macro- and micro-patterns, and more beneficially when stressor and testing is applied to the muscle group after the muscles in one or more higher priority pattern is first stressed according to embodiments of the present disclosure. Further, the criteria of maximum benefit for a muscle group is achieved when stress and testing is applied to the muscle in the heretical order of two or more higher priority patterns prior to the muscle pattern within which the target muscle resides, more beneficially three or more patterns, etc. until all of the muscles within all of the higher priority patterns have been activated or locked-in. So for example, if a secondary muscle in pattern 7 is the target muscle (e.g., injured), the muscles within the first 6 patterns and then within pattern 7 would first be compound stressed or tested prior to activation (bilaterally).

Note also that the disclosure herein also contemplates a process where, using the example above, the first 6 pattern's primary and secondary muscles are treated in order (after a stress), bilaterally, followed by the 7 group's primary muscle and then hierarchy of secondary muscles within pattern 7, in order, including the target muscle.

As discussed above, there are two types of stress application, general and specific. As discussed above, specific stress application is established for each of the 43 muscle pattern's based on a muscle pattern's primary function. One such specific stress application is accomplished by a Muscle Specific Applied Stress (MSAS). MSAS is described in detail in PCT/US16/21607 (Methods, Systems and Kits for Enhanced Muscle Contractile Capabilities), which is incorporated herein by reference for all purposes. The following description focuses on general stress application in combination with MSAS, or compound stress application.

A general stress application in accordance with the present disclosure is through exercise. In most aspects, the exercise is based either on the specific function of the muscle groups or on an individual's gait or gait mechanics. A general stressor is applied by a health care professional. A health care professional for purposes herein refers to licensed and non-licensed providers and includes: medical doctors, doctors of osteopathy, doctors of chiropractic, doctors of physical therapy, massage therapists, nurses, trainers, strength and conditioning coaches and the like.

The inventor's philosophy recognizes that certain general stressors can be used to negatively affect the contractile capabilities of any of the above discussed muscle patterns. This is particularly relevant for the aging process, where, as we age, we become more dysfunctional in our movement patterns. An aging subject's movement pattern becomes less effective in function, and the muscle's ability to contract effectively during the gait movement process is altered. By using gait, in combination with a MSAS, a muscle's weakness can be exposed via AMC&S testing, and undergo corresponding treatment using the hierarchy within the 43 muscle patterns (see below and Table 2). The treatment is highly effective as it both treats the subject's muscles to be more functionally effective as well as ties the subject's muscle groups into the subject's central pattern generator, which orchestrates the rhythmic movement patterns necessary for the subject's gait. As such, a compound stressor both improves muscle function and improves the mechanics of that subject's gait (and limits the dysfunctions of a gait associated with aging).

By using a subject's gait as a general stressor in the compound stress, repeated muscle activation, testing and treatment, ultimately trains the muscles to move from a dysfunction gait pattern to a functional gait pattern, which is then reinforced each time the subject utilizes the function of gait.

Although not wishing to be held to any one theory, Applicant believes that the repetitive nature of compound stress, testing, and treating, ties the muscles required to accomplish a functional gait pattern to a deep network of neurons in the spinal cord called the Central Pattern Generator. The rhythmic movements that occur with gait, or other like general stressor, are governed by the Central Pattern Generator in the spinal cord. With continued performance of the embodiments therein, the programming in the Central Pattern Generator, and thereby the motor neurons, is modified to instruct the muscles to a more functional gait pattern. As this relates to aging, the embodiments herein allow for the reprogramming of the muscle patterns to a functional gait pattern, thereby stopping, if not reversing, the downward progression of muscle function or dysfunction associated with aging.

As such, and in one embodiment, a general stressor is combined with a specific stressor. The general stressor, for example, gait mechanics, is applied in a consistent manner for a subject in need thereof. The primary focus in this embodiment is to stress the subject's muscle pattern through implementation of a high speed walk on a treadmill. The high speed pace of the walk, takes away the subject's ability to compensate for the weaknesses during gait, i.e., a dysfunctional gait, thereby exposing the weak links that have led to the compensatory motions associated with their normal gait pattern.

For purposes herein, a high speed walk, is a walk that on a scale of 1 to 10, with 1 being a slow stroll and 10 being a jog, would constitute a 5 or faster, and in some cases a 6, 7 or 8 or faster. The speed of the walk is somewhat dependent on the subject, but in most instances is meant to require enough stress be put on the subject's muscles, that the weak muscles cannot compensate for a weakened or dysfunctional muscle. A dysfunctional gait pattern for purposes herein is a gait pattern that, when compared to a subject's normal gait pattern or a standardized gait, results in pain, diminished power, diminished strength, or a gait pattern outside the typical two period pattern (double limb support and single limb support). In typical embodiments, the high speed walk is performed for 15 seconds to 2 minutes and more typically from 30 seconds to one minute, or until it is clear that the subject's muscles have been stressed sufficiently to avoid one muscle compensating for any other muscle. In some aspects, the speed of the walk is increased over a period of about 15 seconds to one minute until the subject can no longer maintain the walking gait pattern (typically performed on a treadmill that the subject and/or health professional can control).

Also for purposes herein, not all gait stressors need be performed on a treadmill. In some cases, in which there is a significant weakness in the clients muscular system, performing a gait around the office, track, hallway, etc. is sufficient to establish the required stress.

Once a subject undergoes the gait stressor, he or she is promptly challenged by the muscle specific stress (MSAS) as established by the 43 muscle patterns. In order to determine whether a muscle is showing weakness based on the compound stress, several assessment techniques can be used. In one embodiment, Active Muscle Contract and Sustain Test (AMC&S) is utilized to identify a target muscle's weakness. Note that the MSAS and AMC&S are performed in conjunction with each other, and can be altered such that embodiments contemplate: general stressor, MSAS, AMC&S and treatment, or general stressor, AMC&S, MSAS and treatment. Both AMC&S and MSAS are muscle specific and based on the same 43 muscle pattern.

Typically a AMC&S is a muscle testing technique that is specific to embodiments described herein. AMC&S involves a specific force application of a specific magnitude and rate of force application, set-up and delivered by a health care specialist (see Example 2). The health care specialist assesses the target muscle's ability to react to and meet that force. AMC&S is not a manual muscle “break” test or manual muscle test used as an indication of the body's response to a chemical substance, nor a change in its energetic field, nor a positional post isometric relaxation technique. AMC&S testing is performed in order of the 43 patterns as established in Table 1.

In accordance with the present disclosure, after an appropriate compound stressor period, AMC&S is promptly initiated by the health care practitioner placing the subject in the proper testing position for muscle pattern 1, for example. The subject must relax and then hold the muscle against an applied stress with maximal effort. The health care practitioner takes care not to force the target muscle in multiple directions and should only use passive motion in the plane that matches the applied testing force (for example, adduction on posterior tibialis). It is also important that the subject utilize unconscious control as much as possible and avoid consciously interfering with the muscle's reaction to the applied stress through compensatory motion. Note that the subject's testing position is dictated by his or hers available Range of Motion (“ROM,”) therefore the testing positions will be different with each subject. This may require the health care professional to challenge all testing positions in a pattern (Example 2).

In one embodiment herein, prior to the assessment, a Passive Comparative Assessment of Mobility (CAM) is performed. CAM is a range of motion assessment that is specific to embodiments disclosed herein. CAM is a specific force application leading to the measurement of active or passive limb motion from a designated start position/posture, through a designated plane and direction, to the end of the limb motion. The measurement is then compared to the mirror image limb motion for the limb on the opposite side of the body. CAM is not a joint range of motion examination performed to evaluate passive tissue stability, joint surface pathology, ligamentous integrity, etc. CAM is typically used on a first visit to a health care professional prior to the AMC&S. However, CAM is optional for all other embodiments as described herein.

Once a subject is shown to be properly stressed by the compound stress, and specific muscles locked in, and the weak muscles are identified through use of AMC&S via the predetermined muscle patters, the embodiments herein contemplate treatment of said muscles via a Digital Force Application To Muscle Attachment Technique (DFAMAT) or a Positional Isoangular Contraction (PIC) technique (Table 3).

Typically a treatment in PIC is an activation technique specific to embodiments described herein. PIC involves a specific limb position/orientation (based on the macro and micro patterns shown and discussed herein) and direction of motion generated by the subject, into a barrier to that motion, set-up and maintained by a health care specialist. The health care specialist may use their hands and body to hold/guide limb orientation/positions and provide the barriers to motion during the isoangular contraction. PIC is not a muscle energy technique, strain/counter-strain technique or a post isometric relation technique.

Typically a treatment through DFAMAT is also an activation technique specific to embodiments described herein. DFAMAT involves a specific force application to a target muscle using the health care professional's fingers. The health care professional applies direct pressure perpendicular to a target muscle's attachment (tendons, aponeuroses) using the tips of the fingers instituting motion creating subtle tension on the attachment tissues, followed with motion lines that are perpendicular to each other, maintaining the tension for a duration of 1 to 4 seconds per site, and more typically 1 to 2 seconds per site, releasing and then re-initiating the process, moving along the width/length of the target muscle attachment. DFAMAT is not a soft tissue evaluation nor a manipulation to release trigger points, adhesions, Active Release Technique, move body fluids to and from tissue sites, etc.

Note that DFAMAT is not used to evaluate and interpret the state of soft tissues, nor to create a relaxation response for the target muscle. The premise of DFAMAT is that it stimulates sensory receptors that in turn increase motor neuronal pool activation to the target muscle associated with the attachment. This represents the opposite effect that most, if not all, massage techniques are attempting to achieve as an outcome (relaxation).

In accordance with the present disclosure and DFAMAT, a treatment is applied to the target muscle via palpation to the relevant bone where the target muscle is attached. Identification of the target muscle attachment point requires specific palpation such that as micro-pattern of muscles is being tested then re-tested. Other treatment procedures for increasing a target muscle's set point include isometric and isotonic contractions and the like. Treatments may also include the injection (e.g. intramuscular, intradermal, intravenous) or ingestion of an appropriate biologic.

Biologics contemplated for use herein on an activated or locked-in muscle include: autologous and non-autologous stem cells, anti-inflammatories including anti-rheumatic drugs, immunosuppressants like Methotrexate and azathioprine, anti-cytokines to reduce inflammation like anti-Tumor Necrosis Factor (anti-TNF), medications to repair muscle damage like steroids, platelet-rich plasma or bone-marrow aspirate, chemotherapeutics for treatment of cancer residing in the muscle, for example melanoma (Alemtuzumab, for example), and the like.

Once a subject has undergone a general stress, AMC&S test, MSAS and appropriate treatment according to the predetermined 43 muscle patterns (bilateral), the subject can be treated likewise over a course of days, weeks or years. In some embodiments, the combination of general stress, MSAS, testing and treatment can be performed once a day, twice a week, or once a week, for example. The gait stress, in combination with the MSAS, testing and treatment regiments described herein, tie the subject's muscles into the subject's CPG.

In order to maximize the effect of the general stressor, the gait stress may be performed by the subject between the testing and treatment of each muscle pattern, so for example, a subject may perform a high speed walk for 30 seconds, have AMC&S testing, MSAS, and DFAMAT treatment performed on the muscles, bilaterally, on the first muscle pattern (1). Once complete, the subject would be required to perform a second high speed walk for another 30 seconds, have AMC&S testing, MSAS, and DFAMAT treatment performed on the muscles in accordance with the second muscle pattern (bilaterally). This process of gait stress, MSAS, testing and treatment could be performed on each muscle pattern, 1-43, or according to the patterns previously discussed above (for example, where a returning client has come in, moving from pattern 1 to 7 to 8 to 30).

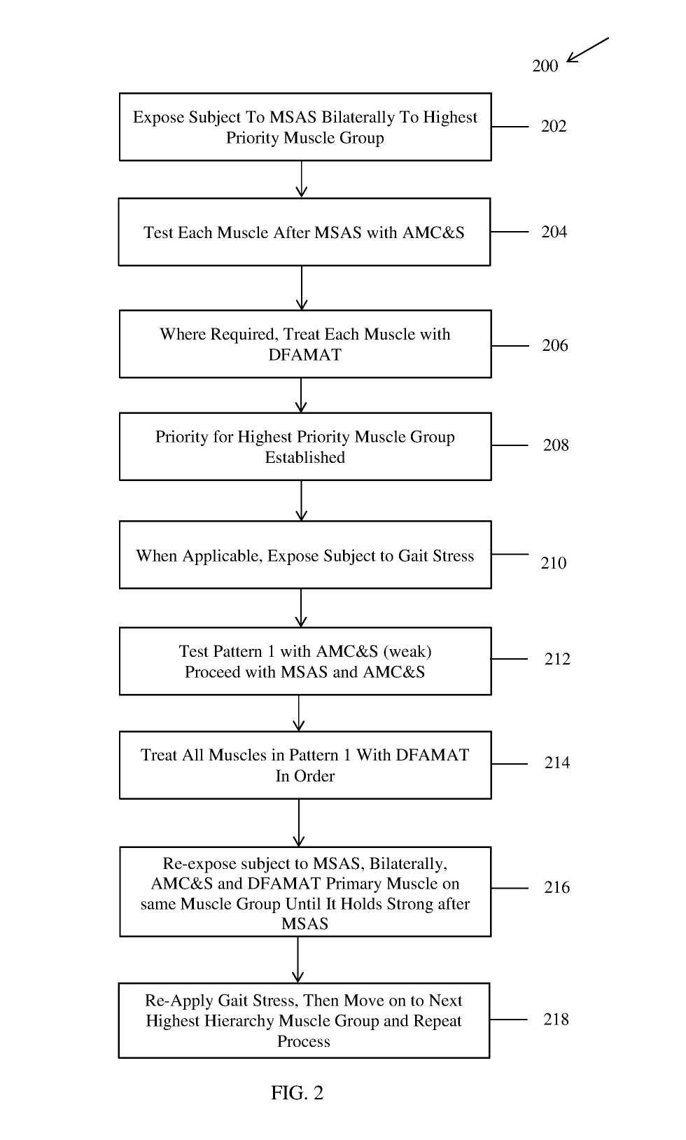

In another example, and as shown in FIG. 2, and in some aspects shown herein, gait mechanics are facilitated in a subject 200 by exposing the subject to MSAS for a highest priority muscle pattern, one for example, bilaterally 202. The muscles are tested by AMC&S 204 and, where required, treated using DFAMAT 206. For this subject, the combination of specific stress, testing and treatment establishes the priority for the pattern 208. The entire muscle pattern can be performed, bilateral macro or bilateral micro, using MSAS, AMC&S and DFAMAT.

In some embodiments, and where applicable, the subject is then exposed to an escalating speed treadmill for 15 seconds to 2 minutes and more typically 30 seconds to one minute. In some embodiments, the goal of the treadmill stressor is to keep the subject from keeping up with the treadmill speed 210. The subject is then tested using AMC&S (weak) 212 specifically stressed using MSAS and treated using DFAMAT 214 over the same previously used MSAS, AMC&S and DFAMAT pattern. The subject would then have the next highest priority muscle group undergo the same iteration of Gait stress, AMC&S, MSAS, and DFAMAT. This pattern would hold for all 43 muscle patterns or any combination of the 43 muscle patterns (in correct hierarchy).

In some circumstances, where the health care professional feels the need, the challenged muscle pattern is again stressed by MSAS, tested, and treated 216. In this embodiment, the subject would undergo (in muscle pattern hierarchy): MSAS, AMC&S, DFAMAT, prior to moving on to the next muscle pattern. This re-exposure will be performed until the MSAS holds strong.

When the muscles in the muscle pattern have been sufficiently treated, he or she may always move onto the next muscle pattern. In this aspect, the gait stress may be re-applied 218. By combining the specific and general stressors, the subject will have trained his or her muscles to be in-tune with the Central Pattern Generator, and thereby begin the process of moving their gait to a more functional gait pattern.

The combination of general stressor, specific stressor, testing, treatment through the hierarchy of the 43 muscle patterns provides an excellent tie for facilitating and correcting a subject's gait patterns and overall functional capabilities. This methodology has proven particularly effective at correcting and improving gait patterns in subjects that have dysfunction gait mechanics due to aging or are dysfunction due to injury.

In an additional embodiment, the muscle pattern need not strictly follow 1-43, but could include skipped muscles as long at the pattern is performed in the previously described 1-43 direction, 2, 8, 16, 35 . . . and not the opposite direction, 37, 28, 14, 2, for example. Also note, a general stressor is always combined with a muscle specific stressor. A muscle specific stressor need not always be combined with a general stressor. So for example, in some embodiments, a health care professional may determine that muscle patterns 1-7 only require MSAS, AMC&S and DFAMAT. At muscle patterns 8 and 9, the health care professional may add the general stressor to the MSAS, AMC&S and DFAMAT, before returning to the MSAS, AMC&S and DFAMAT pattern for muscle patterns 10-42. Typically, in this embodiment, bilateral muscle weakness in a muscle pattern alerts the health care professional that the general stressor, MSAS, AMC&S and DFAMAT is required (groups 8 and 9 in this example). As such, in some embodiments, MSAS, AMC&S and DFAMAT is performed until bilateral muscle weakness is exposed, and then the muscle pattern is subjected to gait testing, MSAS, AMC&S and DFAMAT. As can be imagined, other testing and treatment techniques can be replaced for AMC&S and DFAMAT as disclosed herein, in these embodiments.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

### Example 1

### Movement Pattern Hierarchy and Tables 2-3 Showing Illustrative Tests

The following hierarchy has been established for the 43 movement patterns as classified for purposes herein. This hierarchy from pattern 1 to 43 represents the macro-pattern:

TABLE 1
(Movement Patterns, Left then Right)

| Group/Pattern | | |
|---|---|---|
| Number | Muscle Pattern Macro-Order | Primary Muscle |
| 1 | Trunk Rotation | Transverse Abdominis - Lower |
| | | Division |
| 2 | Trunk Flexion | Psoas Minor |
| 3 | Hip Flexion | Psoas Major: Lumbar Division |
| 4 | Hip Rotation | Obturator Externus |
| 5 | Spinal Sidebend | Longissimus Thoracis |
| 6 | Downward Rotation of the Scapula | Levator Scapula: Superior Division |
| 7 | Humeral External Rotation | Infraspinatus: Superior Division |
| 8 | Spinal Extension | Intertransversatii: Lumborum |
| 9 | Hip Extension | Gluteus Maximus: Iliac Division |
| 10 | Humeral Extension and Adduction | Latissimus Dorsi: Iliac Division |
| 11 | Humeral Internal Rotation | Subscapularis: Superior Division |
| 12 | Elbow Extension | Triceps Brachii: Medial Division |
| 13 | Upward Rotation of the Scapula | Upper Trapezius: Clavicular |
| | | Division |
| 14 | Humeral Abduction | Supraspinatus: Fossa Division |
| 15 | Protraction of the Scapula | Pectoralis Minor: Inferior Division |
| 16 | Horizontal Adduction | Pectoralis Major: Sternal Division |
| 17 | Elbow Flexion | Brachialis |
| 18 | Hip Adduction | Adductor Magnus: Oblique Division |
| 19 | Hip Abduction | Gluteus Medius: Anterior Division |
| 20 | Knee Extension | Rectus Femoris: Straight Division |
| 21 | Knee Flexion | Semitendinosus |
| 22 | Supination of the Foot | Posterior Tibialis: Fibular Division |
| 23 | Plantarflexion | Medial Soleus |
| 24 | 1^{st} Ray Dorsiflexion | Anterior Tibialis: Tibial Division |
| 25 | Pronation | Peroneus Brevis: Lateral Division |
| 26 | Dorsiflexion | Peroneus Tertius: Lateral Division |
| 27 | 1^{st} Ray Plantarflexion | Peroneus Longus: Metatarsal |
| | | Division |
| 28 | Big Toe Extension | Extensor Hallucis Longus: Fibular |
| | | Division |
| 29 | Toe Extension | Extensor Digitorum Longus: Lateral |
| | | Division |
| 30 | Big Toe Flexion | Flexor Hallucis Longus: Fibular |
| | | Division |
| 31 | Toe Flexion | Flexor Digitorum Longus: Lateral |
| | | Division |
| 32 | Cervical Flexion | Longus Capitis |
| 33 | Cervical Rotation | Multifidus Cervicis: Inferior Fibers |
| 34 | Cervical Extension | Semispinalis Capitis |
| 35 | Cervical Sidebend | Posterior Scalene |
| 36 | Wrist Extension with Abduction | Extensor Carpi Radialis Longus: |
| | | Abductor Division |
| 37 | Wrist Flexion with Abduction | Flexor Carpi Radialis Longus: |
| | | Abductor Division |
| 38 | Forearm Supination | Anconeus: Ulnar Division |
| 39 | Forearm Pronation | Pronator Teres: Humeral Division |
| 40 | Extension and Abduction of the Thumb | Extensor Pollicis Longus: Ulnar |
| | | Division |
| 41 | Flexion and Abduction of the Thumb | Flexor Pollicis Longus |
| 42 | Finger Extension | Extensor Digitorum: Medial |
| | | Division |
| 43 | Finger Flexion | Flexor Digitorum Profundus: Medial |
| | | Division |

Hierarchy within a Movement Pattern (Micro-Pattern) (Bilateral, Left first and then Right):
Pattern 1 (Trunk Rotation):
Transverse Abdominis Lower Division
   Internal Oblique: Anterior Division
   External Olique: Anterior Division
   Semispinalis Thoracis
   Transverse Abdominis: Upper Division
   Sternalis
   4^{th} Rectus: Lateral Division
   4^{th} Rectus: Medial Division
Pattern 2 (Trunk Flexion):
Psoas Minor
   Pyramidalis
   1^{st} Rectus Abdominis
   2^{nd} Rectus Abdominis
   3^{rd} Rectus Abdominis
Pattern 3 (Hip Flexion)
Psoas Major: Lumbar Division
   Psoas Major: Thoracic Division
   Psoas Major: Diaphragmatic Division
   Iliacus Major
   Iliacus Minor
   Tensor Fascia Latae: Posterior Division
   Tensor Fascia Latae: Anterior Division
Pattern 4 (Hip Rotation)
Obturator Externus
   Quadratus Femoris
   Piriformis
   Gemellus Inferior
   Gemellus Superior
   Adductor Minimus
   Obturator Internus
Pattern 5 (Spinal Sidebend)
Longissimus Thoracis
   Longissimus Lumborum
   Internal Obliques: Lateral Division
   External Obliques: Lateral Division
   Iliocostalis Thoracis
   Iliocostalis Lumborum
   Multifidus Thoracis
   Multifidus Lumborum
   Quadratus Lumborum: Spinal Division
   Serratus Posterior: Inferior Division
   Serratus Posterior: Superior Division
   Quadratus Lumborum: Costal Division
Pattern 6 (Downward Rotation of the Scapula)
Levator Scapula: Superior Division
   Levator Scapula: Inferior Division
   Rhomboid Minor
   Rhomboid Major
Pattern 7 (Humeral External Rotation)
Infraspinatus: Superior Division
   Infraspinatus: Superior-Middle Division
   Infraspinatus: Inferior-Middle Division
   Infraspinatus: Inferior Division
   Teres Minor
Pattern 8 (Spinal Extension)
Intertransversarii Lumborum
   Interspinalis Lumborum
   Spinalis Thoracis
   Spinalis Lumborum
   Rotatores Thoracis
   Rotatores Lumborum
Pattern 9 (Hip Extension)
Gluteus Maximus: Iliac Division
   Gluteus Maximus: Sacral Division
   Gluteus Maximus: Coccygeal Division
Pattern 10 (Humeral Extension and Adduction)
Latissimus Dorsi: Iliac Division
   Latissimus Dorsi: Lumbar Division
   Latissimus Dorsi: Thoracic Division
   Teres Major: Inferior Division
   Teres Major: Superior Division
   Tricep Brachii: Long Head
Pattern 11 (Humeral Internal Rotation)
Subscapularis: Superior Division
   Subscapularis: Superior/Middle Division
   Subscapularis: Inferior/Middle Division
   Subscapularis: Inferior Division
Pattern 12 (Elbow Extension)
Triceps Brachii: Medial Division
   Triceps Brachii: Lateral Division
   Articularis Cubiti
Pattern 13 (Upward Rotation of the Scapula)
Upper Trapezius: Clavicular Division
   Upper Trapezius: Scapular Division
   Middle Trapezius
   Lower Trapezius
   Serratus Anterior: Superior Division
   Serratus Anterior: Inferior Division
   Subclavius: Lateral Division
   Subclavius: Medial Division
Pattern 14 (Humeral Abduction)
Supraspinatus: Fossa Division
   Supraspinatus: Spinal Division
   Posterior Deltoid: Medial Division
   Posterior Deltoid: Lateral Division
   Middle Deltoid: Posterior Division
   Middle Deltoid: Anterior Division
   Anterior Deltoid: Acromial Division
   Anterior Deltoid: Clavicular Division
Pattern 15 (Protraction of the Scapula)
Pectoralis Minor: Inferior Division
   Pectoralis Minor: Superior Division
Pattern 16 (Horizontal Adduction)
Pectoralis Major: Sternal Division
   Pectoralis Major: Clavicular Division
   Pectoralis Major: Costal Division
   Bicep Brachii: Long Head
   Bicep Brachii: Short Head
   Coracobrachialis: Inferior Division
   Coracobrachialis: Superior Division
Pattern 17 (Elbow Flexion)
Brachialis
   Brachioradialis: Superior Division
   Brachioradialis: Inferior Division
Pattern 18 (Hip Adduction)
Adductor Magnus: Oblique Division
   Adductor Magnus: Vertical Division
   Adductor Longus: Superior Division
   Adductor Longus: Inferior Division
   Adductor Brevis
   Pectineus
   Gracilis
Pattern 19 (Hip Abduction)
Gluteus Medius: Anterior Division
   Gluteus Medius: Posterior Division
   Gluteus Medius: Anterior Division
   Gluteus Minimus: Anterior Division
   Gluteus Minimus: Posterior Division
Pattern 20 (Knee Extension)
Rectus Femoris: Straight Division
   Rectus Femoris: Reflected Division
   Vastus Intermedius: Medial Division
   Vastus Intermedius: Lateral Division
   Vastus Medialis: Superior Division
   Vastus Medialis: Middle Division
   Vastus Medialis: Inferior Division
   Vastus Lateralis: Superior Division
   Vastus Lateralis: Middle Division
   Vastus Lateralis: Inferior Division
   Articularis Genu
Pattern 21 (Knee Flexion)
Semitendinosus
   Semimembranosus: Lateral Division
   Semimembranosus: Medial Division
   Biceps Femoris: Short Head
   Biceps Femoris Long Head: Fibular Division
   Biceps Femoris Long Head: Tibial Division
   Sartorius
   Popliteus
Pattern 22 (Supination)
Posterior Tibialis: Fibular Division
   Posterior Tibialis: Tibial Division
Pattern 23 (Plantarflexion)
Medial Soleus
   Lateral Soleus
   Lateral Gastroc
   Medial Gastroc
   Plantaris
Pattern 24 (1^{st} Ray Dorsiflexion)
Anterior Tibialis: Tibial Division
   Anterior Tibialis: Interossei Division
Pattern 25 (Pronation)
Peroneus Brevis: Lateral Division
   Peroneus Brevis: Posterior Division
Pattern 26 (Dorsiflexion)
Peroneus Tertius: Lateral Division
   Peroneus Tertius: Anterior Division
Pattern 27 (1^{st} Ray Plantarflexion)
Peroneus Longus: Metatarsal Division
   Peroneus Longus: 1st Cuneiform Division
Pattern 28 (Big Toe Extension)
Extensor Hallucis Longus: Fibular Division
   Extensor Hallucis Longus: Interoseii Division
   Extensor Hallucis Brevis
Pattern 29 (Toe Extension)
Extensor Digitorum Longus: Lateral Division
   Extensor Digitorum Longus: Medial Division
   Extensor Digitorum Brevis
   Dorsal Interoseii 2-5
Pattern 30 (Big Toe Flexion)
Flexor Hallucis Longus: Fibular Division
   Flexor Hallucis Longus: Interosseii Division
   Flexor Hallucis Brevis: 1^{st} Cuneiform Division
   Flexor Hallucis Brevis: Cuboid Division
   Flexor Hallucis Brevis: 3^{rd} Cuneiform/Tensonal Division
   Adductor Hallucis Longus: Oblique Head
   Adductor Hallucis Longus: Transverse Head, Lateral
   Adductor Hallucis Longus: Transverse Head, Medial
   Abductor Hallucis Longus: Invertor Division
   Abductor Hallucis Longus: Adductor Division
Pattern 31 (Toe Flexion)
Flexor Digitorum Longus: Lateral Division
   Flexor Digitorum Longus: Medial Division
   Flexor Digitorum Brevis: Lateral Division
   Flexor Digitorum Brevis: Medial Division
   Quadratus Plantae: Lateral Division
   Quadratus Plantae: Medial Division
   Lumbricals: 5-2
   Plantar Interoseii: 5-3
   Abductor Digiti Minimi
   Flexor Digiti Minimi Brevis
Pattern 32 (Cervical Flexion)
Longus Capitis
   Longus Colli: Superior Oblique Fibers
   Longus Colli: Vertical Fibers
   Longus Colli: Inferior Fibers
   Mylohyoid
   Sternohyoid
   Rectus Capitis Anterior
Pattern 33 (Cervical Rotation)
Multifidus Cervicis: Interior Fibers
   Multifidus Cervicis: Superior Fibers
   Sterno-cleadomastoid: Sternal Fibers
   Sterno-cleadomastoid: Clavicular Fibers
   Longissimus Capitis
   Longissimus Cervicis
   Splenius Capitis: Occipital Fibers
   Splenius Capitis: Mastoid Fibers
   Splenius Cervicis
   Iliocostalis Cervicis
   Rotatores Cervicis
   Rectus Capitis Posterior Major
   Obliques Capitis Inferior
Pattern 34 (Cervical Extension)
Semispinalis Capitis
   Semispinalis Cervicis
   Spinalis Capitis
   Spinalis Cervicis
   Interspinalis Cervicis
   Obliques Capitis Superior
   Rectus Capitis Posterior Minor
Pattern 35 (Cervical Sidebend)
Posterior Scalene
   Middle Scalene
   Anterior Scalene
   Anterior Intertransversarii
   Posterior Intertransversarii
   Omohyoid
   Rectus Capitis Lateralis
Pattern 36 (Wrist Extension with Abduction)
Extensor Carpi Radialis Longus: Abductor Division
   Extensor Carpi Radialis Longus: Extensor Division
   Extensor Carpi Ulnaris Longus: Adductor Division
   Extensor Carpi Ulnaris Longus: Extensor Division
   Extensor Carpi Radialis Brevis
Pattern 37 (Wrist Flexion with Abduction)
Flexor Carpi Radialis Longus: Abductor Division
   Flexor Carpi Radialis Longus: Flexor Division
   Flexor Carpi Ulnaris Longus: Adductor Division
   Flexor Carpi Ulnaris Longus: Flexor Division
   Palmaris Longus
Pattern 38 (Forearm Supination)
Anconeus: Ulnar Division
   Anconeus: Olecranon Division
   Supinator: Olecranon Division
   Supinator: Ulnar Division
Pattern 39 (Forearm Pronation)
Pronator Teres: Humeral Division
   Pronator Teres: Ulnar Division
   Pronator Quadratus: Proximal Division
   Pronator Quadratus: Distal Division
Pattern 40 (Extension and Abduction of the Thumb)
Extensor Pollicis Longus: Ulnar Division
   Extensor Pollicis Longus: Septal Division
   Extensor Pollicis Brevis: Radial Division
   Extensor Pollicis Brevis: Septal Division
   Abductor Pollicis Longus: Radial Division
   Abductor Pollicis Longus: Ulnar Division
Pattern 41 (Flexion and Abduction of the Thumb)
Flexor Pollicis Longus
   Abductor Pollicis Brevis
   Flexor Pollicis Brevis
   Adductor Pollicis: Oblique Head
   Adductor Pollicis: Transverse Head
   Interosseus Pollicis
   Opponens Pollicis: Flexor Division
   Opponens Pollicis: Abductor Division
Pattern 42 (Finger Extension)
Extensor Digitorum: Medial Division
   Extensor Digitorum: Lateral Division
   Extensor Indicis
   Extensor Digiti Minimi
   Dorsal Interoseii: 1-4
Pattern 43 (Finger Flexion)
Flexor Digitorum Profundus: Medial Division
   Flexor Digitorum Profundus: Lateral Division
   Flexor Digitorum Superficialis: Medial Division
   Flexor Digitorum Superficialis: Lateral Division
   Lumbricals: 4-1
   Palmar Interoseii: 4-2
   Flexor Digiti Minimi
   Abductor Digiti Minimi: Flexor Division
   Abductor Digiti Minimi: Abductor Division
   Oponens Digiti Minimi Manus: Flexor Division
   Oponens Digiti Minimi Manus: Abductor Division
   Palmaris Brevis

TABLE 2
Illustrative DFAMAT

| DFAMAT Pattern | | | |
|---|---|---|---|
| Number/Muscle | Origin | Insertion | Tips |
| 1 (Transverse | Thoraco-lumbar | Linea alba below | Spinous process of |
| Abdominis: Lower | fascia, anterior ¾ of | umbilicus and into the | T12-L5 and sacrum |
| Fibers) | iliac crest and lateral | pubic symphysis | Anterior ¾ of iliac |
| | inguinal ligament | | crest |
| | | | Inguinal ligament |
| | | | Superior to pubic |
| | | | bone |
| | | | Up linea alba to |
| | | | umbilicus |
| 2 (Internal Obliques: | Lateral ⅔ of inguinal | With transverse | Sidelying, palpate |
| Anterior Fibers) | ligament and anterior | abdominis into crest | xyphoid process, |
| | iliac crest | of pubis and into linea | down linea alba to |
| | | alba through an | umbilicis. |
| | | aponeurosis | Palpate from xyphoid |
| | | | process down and out |
| | | | along costal cartilage |
| | | | of ribs |
| | | | Palpate anterior ¾ of |
| | | | superior iliac crest |
| | | | Palpate spinous |
| | | | processes of T12-L5 |
| 3 (Exterior Obliques: | Interdigitates surface | Into linea alba | Sidelying, use |
| Anterior Fibers) | of ribs 5-8 | through aponeurosis | xyphoid process as |
| | | | landmark, move up |
| | | | and over to 5^{th} rib, |
| | | | anterior to the serratus |
| | | | Palpate anterior aspect |
| | | | of ribs 5-8; angling |
| | | | back towards the |
| | | | spine |
| | | | Palpate |
| | | | superior/anterior ½ of |
| | | | ilium to ASIS |
| | | | Supine, palpate down |
| | | | aponeurosis and |
| | | | inguinal ligament |
| 4 (Semispinalis | Arise from transverse | 1-10 thoracic and | Spinous processes |
| Thoracis) | process of all thoracic | lower 4 cervical | from C4 down to T10 |
| | vertebrae | spinous processes | Palpate transverse |
| | | | processes from C7- |
| | | | T1 to T12 |
| 5 (Transverse | Cartilage of lower 6 | Linea alba superior to | Supine, palpate |
| Abdominis: Upper | ribs | umbilicus | xyphoid process, |
| Fibers) | | | palpate along cartilage |
| | | | of ribs to angle |
| | | | Down linea alba from |
| | | | xyphoid to umbilicus |
| 6 (Sternalis) | Manubrium and | Superior medial fascia | Supine, palpate at |
| | inferior-medial | of 4^{th} rectus | inferior-medial |
| | clavicle | | clavicle and moving 1 |
| | | | inch lateral on |
| | | | clavicle |
| | | | Palpate superior- |
| | | | medial portion of 4^{th} |
| | | | section of the rectus |
| | | | abdominis |
| 7 (Rectus Abdominis; | From 3^{rd} section of | Into costal-cartilage of | Supine, use xyphoid |
| Fourth Section | rectus abdominis | 6^{th} and 7^{th} rib | process as landmark, |
| Lateral) | | | move across to |
| | | | anterior portion of 6^{th} |
| | | | & 7^{th} rib |
| | | | Palpate down lateral |
| | | | aponeurosis and |
| | | | across inferior |
| | | | attachment |
| | | | Palpate up linea alba |
| | | | to xyphoid process |
| 8 (Rectus Abdominis: | From 3^{rd} section of | Into cartilage of 5^{th} rib | Supine, use xyphoid |
| Fourth Section: | rectus abdominis: | and side of xyphoid | process as landmark, |
| Medial) | lateral half | process | move across and up to |
| | | | lower level of 5^{th} rib |
| | | | against sternum |
| | | | Palpate down lateral |
| | | | aponeurosis and |
| | | | across inferior |
| | | | attachment |
| | | | Palpate up linea alba |
| | | | to xyphoid process |
| 9 (PSOAS MINOR) | Anterior-lateral | The pectineal line, the | Xyphoid process to |
| | bodies of T12 & L1 | ilio-pectineal | anterior body of T12, |
| | (L2) vertebrae and | eminence and the iliac | L1 & L2 |
| | associated disc | fascia | Move down, palpate |
| | | | deep to superior |
| | | | ramus of pubis and |
| | | | inguinal ligament |
| 10 (Pyramidalis) | Front of pubis and | Linea alba midway | Supine, palpate |
| | anterior pubic | between pubic bone | superior medial pubic |
| | ligament | and umbilicus | bone |
| | | | Up linea alba 1/3 up |
| | | | toward umbilicus |
| | | | Angle downward to |
| | | | lateral pubis |
| | | | Always palpating into |
| | | | muscle belly when |
| | | | working abdominal |
| | | | wall |
| 11 (Rectus | Inner origin of 2^{nd} | Pubic crest and | Supine, use umbilicus |
| Abdominis: First | section of rectus | symphysis. Also | as a landmark, find |
| Division) | abdominis | lateral expansion to | fascial line just below |
| | | opposite side | Palpate across to |
| | | | aponeurosis |
| | | | Follow downward to |
| | | | pubic bone |
| | | | Across pubic bone |
| | | | and back up linea alba |
| | | | Always palpating into |
| | | | muscle belly when |
| | | | working abdominal |
| | | | wall |
| 12 (Rectus | From 1^{st} section of | Into 2^{nd} section of | Find bottom of 2^{nd} |
| Abdominis: Second | rectus abdominis | rectus abdominis | level |
| Division) | | | Palpate across, up side |
| | | | of aponeurosis |
| | | | Palpate for superior |
| | | | fascia, palpate across |
| | | | and down linea alba |
| | | | Always palpating into |
| | | | muscle belly when |
| | | | working abdominal |
| | | | wall |
| 13 (Rectus | From 3^{rd} section of | Into 4^{th} section of | Supine, use umbilicus |
| Abdominis: Third | rectus abdominis | rectus abdominis | as landmark, find top |
| Section) | | | of 2^{nd} level |
| | | | Palpate across, up side |
| | | | of aponeurosis |
| | | | Palpate for superior |
| | | | fascia, palpate across |
| | | | and down linea alba |
| | | | Always palpating into |
| | | | muscle belly when |
| | | | working abdominal |
| | | | wall |
| 14 (PSOAS Major: | Bodies and Anterior | Lesser trochanter of | Supine, flex involved |
| Lumbar Fibers) | surface of transverse | the femur | hip by bending knee |
| | processes of L2-L5 | | and have client exhale |
| | | | Small circles to move |
| | | | abdominal contents to |
| | | | the side |
| | | | Active hip flexion to |
| | | | confirm |
| | | | Palpate L2-L5 |
| | | | transverse processes |
| | | | and bodies |
| | | | Palpate above |
| | | | adductor longus |
| | | | tendon into lesser |
| | | | trochanter |
| 15 (PSOAS Major | Bodies and transverse | Lesser trochanter of | Supine, flex involved |
| Thoracic Fibers) | processes of T12 & | the femur | hip and have client |
| | L1 | | exhale |
| | | | Small circles to move |
| | | | abdominal contents to |
| | | | the side |
| | | | Active hip flexion to |
| | | | confirm |
| | | | Palpate T12 & L1 |
| | | | transverse processes |
| | | | and bodies |
| | | | Palpate above |
| | | | adductor longus |
| | | | tendon into lesser |
| | | | trochanter |
| 16 (PSOAS Major: | Right crus: upper 3 | Central tendon; mid- | Supine, palpate from |
| Diaphragmatic Fibers) | lumbar bodies | central part of | xyphoid process to rib |
| | Left crus: upper 2 | xyphoid process | cage |
| | lumbar bodies | | Have patient exhale to |
| | | | relax diaphragm and |
| | | | deflate lungs |
| | | | Press fingers into |
| | | | where diaphragm |
| | | | connects with thorax |
| | | | Palpate diaphragm |
| | | | down to angle of |
| | | | ribcage |
| | | | Palpate associated |
| | | | lumbar bodies |
| 17 (Iliacus) | Anterior surface of | Lesser trochanter of | Supine, client flexes |
| | iliac crest | femur | hip with femur |
| | | | externally rotated |
| | | | Curl fingers into iliac |
| | | | fossa |
| | | | Confirm through |
| | | | active hip flexion |
| | | | Palpate above |
| | | | adductor longus |
| | | | tendon into lesser |
| | | | trochanter |
| 18 (Iliacus Minor) | Anterior surface of | Lesser trochanter of | Client flexes hip with |
| | iliac crest | femur | femur externally |
| | | | rotated |
| | | | Curl fingers into iliac |
| | | | fossa palpating |
| | | | superficial belly |
| | | | Palpate above |
| | | | adductor longus |
| | | | tendon into lesser |
| | | | trochanter |
| 19 (Tensor Fascia | Anterior portion of | Into ilio-tibial tract | Supine, up and into |
| Latae Posterior | outer lip of iliac crest | just below joint | iliac crest posterior to |
| Fibers) | | capsule | ASIS |
| | | | Palpate at insertion |
| | | | into IT-Tract |
| | | | Superior portion of |
| | | | lateral condyle |
| | | | Palpate insertion of |
| | | | IT-band into lateral |
| | | | condyle of tibia |
| 20 (Tensor Fascia | Anterior portion of | Into ilio-tibial tract | Supine, up and into |
| Latae Anterior Fibers) | outer lip of iliac crest | just below joint | iliac crest just off |
| | | capsule | ASIS |
| | | | Palpate at insertion |
| | | | into IT-tract |
| | | | Superior portion of |
| | | | lateral condyle |
| | | | Palpate insertion of |
| | | | IT-band into lateral |
| | | | condyle of tibia |

### Example 2

### Illustrative AMC&S Tests

1. Micro-Order 1, AMC&S Test

Transverse Abdominis Lower Fibers

Tester Position:
Body: Stand on uninvolved side
Stabilizing Hand: posterior-lateral side of involved ilium to hold end range spinal rotation
Action Hand: contact on medial side of involved knee
**maintain endrange rotation

Client Position:
Supine, Anchor thorax by wrapping arms around top of table
Head in headpiece
Flex hip to 90 degrees, with knee slightly flexed.
Cross lag across uninvolved thigh to create spinal rotation

Applied Force:
Maintain spinal rotation through stabilizing hand until testing force is applied
Counter rotation force through involved leg to create opposite side spinal rotation

Internal Oblique: Anterior

Tester Position:
Body: Stand on involved side
Leg across thigh
Stabilizing Hand: Grab uninvolved shoulder
Action Hand: anterior side of involved shoulder just below clavicle
**maintain end range rotation and neutral spine

Client Position:
Supine, Flex trunk to 90 degrees with feet on the table and knees slightly bent
Feet shoulder width apart
Cross arms across chest and raise elbows to 90 degrees of shoulder flexion in order to maintain spinal neutral
Fully rotate trunk to involved side while maintaining spinal neutral

Applied Force:
Extension through same side rotation

External Oblique: Anterior AMC&S Test

Tester Position:
Body: Stand on uninvolved side
Leg across thigh
Stabilizing Hand: Grab involved shoulder
Action Hand: anterior side of involved shoulder just below clavicle
**maintain end range rotation and neutral spine

Client Position:
Supine, flex trunk to 90 degrees with feet on the table and knees slightly bent
Feet shoulder width apart
Cross arms across chest and raise elbows to 90 degrees of shoulder flexion in order to maintain spinal neutral
Fully rotate trunk to opposite side while maintaining spinal neutral

Applied Force:
Counter-rotation

Semispinalis Thoracis

Tester Position:
Body: Stand on same side of muscle being tested
Leg over posterior hip across to involved side
Grab anterior aspect of both shoulders to assist in extension and opposite rotation
Stabilizing Hand: Maintain end range extension and rotation by holding uninvolved side shoulder up
Action Hand: move hand to posterior shoulder on uninvolved side

Client Position:
Prone, arms to side
Extend and fully rotate thorax to opposite side

Applied Force:
Counter-rotation

Transverse Abdominis: Upper

Tester Position:
Body: Stand on uninvolved side
Leg across thigh
Stabilizing hand: under mid-thoracic spine to assist in extension and rotation of spine
Action Hand: Grab posterior aspect of involved shoulder to maintain opposite rotation: shift to anterior side of involved shoulder just below clavicle

Client Position:
Supine, feet shoulder width apart
Cross arms across chest
Flex trunk 20 degrees with feet on table
Fully rotate trunk to opposite side while maintaining neutral position of the spine

Applied Force:
Counter-rotation

Sternalis

Tester Position:
Body: Stand on uninvolved side
Leg across thigh
Stabilizing hand: under mid-thoracic spine to assist in opposite rotation
Action Hand: Grab posterior aspect of involved shoulder to maintain opposite rotation: shift to anterior side of involved shoulder just below clavicle

Client Position:
Supine, feet on the table and knees slightly bent
Feet shoulder width apart
Cross arms across chest
Crunch up through thoracic spine to flex trunk 20 degrees
Fully rotate trunk to opposite side
Fully exhale

Applied Force:
Counter-rotation force

Rectus Abdominis: 4th Lateral

Tester Position:
Body: Stand on uninvolved side
Leg across thigh
Stabilizing hand: under mid-thoracic spine to assist in extension and rotation of spine
Action Hand: Grab posterior aspect of involved shoulder to maintain opposite rotation: shift to anterior side of involved shoulder just below clavicle

Client Position:
Supine, feet on the table with knees slightly bent
Feet shoulder width apart
Cross and raise arms from chest
Flex trunk 45 degrees
Fully rotate trunk to opposite side while maintaining spinal neutral

Applied Force:
Counter-rotation

Rectus Abdominis: 4th Medial

Tester Position:
Body: Stand on involved side
Leg across thigh
Stabilizing hand: under mid-thoracic spine to assist in extension of spine
Action Hand: Anterior side of involved shoulder just below clavicle

Client Position:
Supine, with feet on the table and knees slightly bent
Feet shoulder width apart
Cross and raise arms from chest
Flex trunk 45 degrees
Slightly rotate trunk to opposite side while maintaining spinal neutral

Applied Force:
Extension

2. Micro-Order 2, AMC&S Test

Psoas Minor

Tester Position:
Body: Stand on involved side
Stabilizing Hand: Stabilize under involved glute/sacrum in order to maintain posterior pelvic tilt
Action Hand: wrap around ankle

Client Position:
Supine, Anchor thorax by wrapping arms around top of table
Externally rotate involved leg
Flex and slightly adduct hip toward midline of the body (maintaining external rotation of the femur) to drive a posterior pelvic tilt and spinal flexion

Applied Force:
Extend and slightly abduct thigh through oblique plane to create anterior pelvic tilt and spinal extension

Pyramidalis

Tester Position:
Body: Stand on involved side
Leg across shin**
Stabilizing Hand: stabilize across back guiding thorax into end range of trunk and spinal flexion
Action Hand: anterior side of involved shoulder just below clavicle

Client Position:
Supine, with feet on the table and knees slightly bent
Feet shoulder width apart
Reach arms behind back
Fully flex trunk and spine
Slightly rotate trunk to opposite side

Applied Force:
Extension

Rectus Abdominis: 1st

Tester Position:
Body: Stand on involved side
Leg across thigh
Stabilizing Hand: stabilize across back guiding thorax into end range of spinal flexion
Action Hand: anterior side of involved shoulder just below clavicle

Client Position:
Supine, with feet on the table and knees slightly bent:
Feet shoulder width apart
Cross arms across chest
Flex trunk 90 degrees
Slouch shoulders downwards to enhance spinal flexion
Slightly rotate trunk to opposite side

Applied Force:
Extension

Rectus Abdominis: 2nd

Tester Position:
Body: Stand on involved side
Leg across thigh
Stabilizing Hand: stabilize across back guiding thorax to maintain spinal neutral
Action Hand: anterior side of involved shoulder just below clavicle

Client Position:
Supine, with feet on the table and knees slightly bent
Feet shoulder width apart
Cross and raise arms from chest
Flex trunk 90 degrees
Slightly rotate trunk to opposite side while maintaining spinal extension

Applied Force:
Extension

Rectus Abdominis: 3rd

Tester Position:
Body: Stand on involved side
Leg across thigh
Stabilizing Hand: stabilize across back guiding thorax to maintain spinal neutral
Action Hand: anterior side of involved shoulder just below clavicle

Client Position:
Supine, with feet on table and knees slightly bent
Feet shoulder width apart
Cross and raise arms from chest
Flex trunk 75 degrees
Slightly rotate trunk to opposite side while maintaining spinal neutral

Applied Force:
Extension

3. Micro Order 3, AMC&S Test

Psoas Major: Lumbar Fibers

Tester Position:
Body: Stand on involved side
Stabilizing Hand: Stabilize anterior side of involved thigh to maintain knee extension
Move to opposite ASIS
Action Hand: grab lower leg at ankle

Client Position:
Supine, Anchor thorax by wrapping arms around top of table
Externally rotate involved leg
Fully abduct leg, maintaining external rotation and knee extension
Flex and adduct leg through oblique plane toward opposite thorax

Applied Force:
Hip Extension and abduction through oblique plane

Psoas Major: Thoracic Fibers

Tester Position:
Body: Stand on involved side
Stabilizing Hand: Stabilize anterior side of involved thigh to maintain knee extension, switch to opp ASIS
Action Hand: grab lower leg at ankle

Client Position:
Supine, Anchor thorax by wrapping arms around top of table
Externally rotate involved leg
Fully abduct leg, maintaining external rotation and knee extension
Flex hip 30 degrees

Applied Force:
Hip Extension

Psoas Major: Diaphragmatic

Tester Position:
Body: Stand on involved side
Stabilizing Hand: Stabilize anterior side of involved thigh to maintain knee extension: switch to opp ASIS
Action Hand: grab lower leg at ankle

Client Position:
Supine, Anchor thorax by wrapping arms around top of table
Externally rotate involved leg
Fully abduct leg, maintaining external rotation and knee extension
Flex hip 10 degrees

Applied Force:
Hip Extension

Iliacus

Tester Position:
Body: Stand on involved side
Leg across thigh
Stabilizing Hand: Stabilize anterior side of involved thigh to maintain knee extension
Action Hand: grab lower leg at ankle

Client Position:
Supine, Anchor thorax by wrapping arms around top of table
Externally rotate involved leg
Fully flex hip maintaining external rotation and knee extension

Applied Force:
Hip Extension

Iliacus Minor

Tester Position:
Body: Stand on uninvolved side*
Stabilizing Hand: Stabilize anterior side of involved thigh to maintain knee extension
Action Hand: grab lower leg at ankle

Client Position:
Supine, anchor thorax by wrapping arms around end of table
Externally rotate leg
Fully flex then adduct leg through oblique plane maintaining external rotation and knee extension

Applied Force:
Hip Extension and abduction through the oblique plane

Tensor Fascia Latae: Posterior Fibers

Tester Position:
Body: Stand on involved side
Stabilizing Hand: Stabilize anterior side of involved thigh to maintain knee extension
Action Hand: grab lower leg at ankle

Client Position:
Supine, Anchor thorax by wrapping arms around top of table
Internally rotate involved leg
Fully abduct involved leg, maintaining internal rotation and knee extension
Fully flex hip from abducted position

Applied Force:
Hip Extension

Tensor Fascia Latae Anterior Fibers

Tester Position:
Body: Stand on involved side
Stabilizing Hand: Stabilize anterior side of involved thigh to maintain knee extension
Action Hand: grab lower leg at ankle

Client Position:
Supine, Anchor thorax by wrapping arms around top of table
Internally rotate involved leg
Fully abduct involved leg, maintaining internal rotation and knee extension
Fully flex hip from abducted position

Applied Force:
Hip Extension and adduction through the oblique plane

4. Micro Order 4, AMC&S Test

Obturator Externus

Tester Position:
Body: stand on involved side
Leg across opposite thigh
Stabilizing Hand: lateral side of involved knee
Action Hand: cup involved heel

Client Position:
Supine, Anchor thorax by wrapping arms around top of table
Flex involved hip
Fully externally rotate the femur at the hip with tibia just below parallel to the table

Applied Force:
Internal rotation

Quadratus Femoris

Tester Position:
Body: stand on involved side
Stabilizing Hand: lateral side of involved knee
Action Hand: cup involved heel

Client Position:
Supine, Anchor thorax by wrapping arms around top of table
Flex hip 75°,
Fully externally rotate the femur at the hip. Tibia parallel to the table

Applied Force:
Internal rotation

Piriformis

Tester Position:
Body: stand on involved side
Stabilizing Hand: Involved PSIS
Action Hand: grab involved ankle

Client Position:
Prone, flex knee 90°
Abduct involved thigh to tissue tension
Fully externally rotate the femur at the hip

Applied Force:
Internal rotation

Gemellus Inferior

Tester Position:
Body: stand on involved side
Stabilizing Hand: Involved PSIS
Action Hand: grab involved ankle

Client Position:
Prone, flex knee 90°
Abduct thigh 30°
Drop knee off side of table to create 20° of hip flexion
Brace against thigh
Fully externally rotate the femur at the hip

Applied Force:
Internal rotation

Gemellus Superior

Tester Position:
Body: stand on involved side
Stabilizing Hand: Medial aspect of involved thigh (reach around)
Action Hand: grab involved ankle

Client Position:
Prone, flex knee 90°
Abduct thigh 30°
Drop knee off side of table to create 45° of hip flexion
Fully externally rotate the femur at the hip

Applied Force:
Internal rotation

Adductor Minimus

Tester Position:
Body: stand on involved side
Stabilizing Hand: Involved PSIS
Action Hand: grab involved ankle

Client Position:
Prone, legs straight
Flex knee 90°
Adduct involved leg to tissue tension
Fully externally rotate the femur at the hip

Applied Force:
Internal rotation

Obturator Internus

Tester Position:
Body: stand on involved side
Stabilizing Hand: uninvolved ASIS
Action Hand: lateral side of involved knee

Client Position:
Supine, slide to edge of the table on involved side
Flex knee 100 degrees to level of opposite knee
Place plantar aspect of foot against side of table
Flex, abduct and externally rotate femur at hip

Applied Force:
Flexion and Adduction

5. Micro Order 5, AMC&S Test

Longissimus Thoracis

Tester Position:
Body: Stand on uninvolved side
Stabilizing Hand: stabilize uninvolved thorax, up and in to inferior rib cage
Start by setting pelvis in neutral
Action Hand: reach arm under knees, grabbing inferior-lateral side of involved knee

Client Position:
Supine, slide body to the top of the uninvolved side of the table
Anchor uninvolved side arm around upper corner of table
Sidebend thorax on ilium
Cross involved side arm on chest
Take both legs to involved side to create end range spinal sidebend

Applied Force:
Opposite sidebend
Keep body flat in plane of the table

Longissimus Lumborum

Tester Position:
Body: Stand on uninvolved side
Stabilizing Hand: stabilize uninvolved ilium, up and in to inferior iliac crest
Start by setting pelvis in neutral
Action Hand: reach arm under knees, grabbing inferior-lateral side of involved knee

Client Position:
Supine, slide body to the top of the uninvolved side of the table
Anchor uninvolved side arm around upper corner of table
Sidebend thorax on ilium
Cross involved side arm on chest
Take both legs to involved side to create end range spinal sidebend

Applied Force:
Opposite sidebend
Keep body flat in plane of the table

Internal Oblique: Lateral

Tester Position:
Body: Stand on involved side
Stabilizing Hand: stabilize involved thigh
Action Hand: Lateral side of involved shoulder

Client Position:
Supine, with feet on table with knees slightly bent
Feet shoulder width apart
Cross arms
Flex trunk 90 degrees
Fully rotate trunk to opposite side
Laterally bend at trunk through plane of shoulders toward involved side

Applied Force:
Opposite sidebend: through plane of shoulders

External Oblique: Lateral

Tester Position:
Body: Stand on uninvolved side
Stabilizing Hand: stabilize uninvolved ilium pressing up and in to iliac crest
Action Hand: reach arm under knees, grabbing inferior-lateral side of involved knee

Client Position:
Supine, Cross arms on chest
Elevate legs 20 degrees with knees straight
Take both legs to involved side to create end range spinal sidebend

Applied Force:
Opposite sidebend
Keep body flat in plane of the table

Iliocostalis Thoracis

Tester Position:
Body: Stand on uninvolved side
Stabilizing Hand: stabilize uninvolved thorax pressing up and in to inferior rib cage
Action Hand: reach arm under knees, grabbing inferior-lateral side of involved knee

Client Position:
Supine, legs straight
Internally rotate leg on involved side
Cross arms on chest
Take both legs to involved side to create end range spinal sidebend

Applied Force:
Opposite sidebend
Keep body flat in plane of the table

Iliocostalis Lumborum

Tester Position:
Body: Stand on uninvolved side
Stabilizing Hand: stabilize uninvolved ilium pressing up and in to inferior iliac crest
Action Hand: reach arm under knees, grabbing inferior-lateral side of involved knee

Client Position:
Supine, legs straight
Internally rotate leg on involved side
Cross arms on chest
Take both legs to involved side to create end range spinal sidebend

Applied Force:
Opposite sidebend
Keep body flat in plane of the table

Multifidus: Thoraco-Lumbar

Tester Position:
Body: Stand on uninvolved side
Stabilizing Hand: stabilize uninvolved thorax pressing up and in to inferior rib cage
Action Hand: reach arm under knees, grabbing inferior-lateral side of involved knee

Client Position:
Supine, legs straight
Externally rotate leg on involved side
Cross arms on chest
Take both legs to involved side to create end range spinal sidebend

Applied Force:
Opposite sidebend
Keep body flat in plane of the table

Multifidus: Lumbo-Sacral

Tester Position:
Body: Stand on uninvolved side
Stabilizing Hand: stabilize uninvolved ilium pressing up and in to inferior iliac crest
Action Hand: reach arm under knees, grabbing inferior-lateral side of involved knee

Client Position:
Supine, legs straight
Externally rotate leg on involved side
Cross arms on chest
Take both legs to involved side to create end range spinal sidebend

Applied Force:
Opposite sidebend
Keep body flat in plane of the table

Quadratus Lumborum Spinal Fibers

Tester Position:
Body: Stand on uninvolved side
Stabilizing Hand: stabilize uninvolved ilium pressing up and in to inferior iliac crest
Action Hand: reach arm under knees, grabbing inferior-lateral side of involved knee

Client Position:
Supine, legs straight
Cross arms on chest
Take both legs to involved side to create end range spinal sidebend

Applied Force:
Opposite sidebend
Keep body flat in plane of the table

Serratus Posterior: Inferior

Tester Position:
Body: Stand on involved side
Stabilizing Hand: stabilize involved thigh
Action Hand: Lateral side of involved shoulder

Client Position:
Supine, feet on table with knees slightly bent
Feet shoulder width apart
Cross arms
Flex trunk 90 degrees
Fully rotate trunk to involved side
Laterally bend at trunk through plane of shoulders toward involved side
Fully exhale

Applied Force:
Opposite sidebend: through plane of shoulders

Serratus Posterior: Superior

Tester Position:
Body: Stand on same side of muscle being tested
Leg across hip to brace involved side
Grab anterior aspect of both shoulders to assist in extension and opposite rotation
Stabilizing Hand: Maintain end range extension and rotation by holding involved side shoulder up
Action Hand: move hand to posterior shoulder on uninvolved side

Client Position:
Prone, arms to side
Fully extend and rotate thorax to opposite side
Fully exhale

Applied Force:
Counter-rotation

Quadratus Lumborum Costal Fibers

Tester Position:
Body: Stand on uninvolved side
Stabilizing Hand: stabilize uninvolved ilium pressing up and in to inferior iliac crest
Action Hand: reach arm under knees, grabbing inferior-lateral side of involved knee

Client Position:
Supine, legs straight
Cross arms on chest
Take both legs to involved side to create 10 degrees of spinal sidebend*

Applied Force:
Opposite sidebend
Keep body flat in plane of the table

6. Micro Order 6, AMC&S Test

Levator Scapula Superior Division

Tester Position:
Body: Stand on involved side
Stabilizing Hand: posterior-lateral side of involved wrist to maintain humeral external rotation and downward rotation of the scapula
Action Hand: Medial side of involved elbow

Client Position:
Supine, slide to edge of the table to allow scapula to retract off the side of the table
Rotate head to ipsilateral side
Flex elbow 90 degrees
Externally rotate and adduct humerus
Downwardly rotate the scapula

Applied Force:
Abduct humerus to upwardly rotate the scapula

Levator Scapula Inferior Division

Tester Position:
Body: Stand on involved side
Stabilizing Hand: posterior-lateral side of involved wrist to maintain humeral external rotation and downward rotation of the scapula
Action Hand: Medial side of involved elbow

Client Position:
Supine, slide to edge of the table to allow scapula to retract off the side of the table
Rotate head to ipsilateral side
Flex elbow 90 degrees
Adduct humerus
Downwardly rotate the scapula

Applied Force:
Abduct humerus to upwardly rotate the scapula

Rhomboid Minor

Tester Position:
Body: Stand on involved side
Stabilizing Hand: posterior-lateral side of involved wrist to maintain humeral external rotation and downward rotation of the scapula
Action Hand: Medial side of involved elbow

Client Position:
Supine with elbow flexed to 90 degrees
Abduct humerus 20 degrees
Externally rotate humerus
Downwardly rotate the scapula

Applied Force:
Abduct humerus to upwardly rotate the scapula

Rhomboid Major

Tester Position:
Body: Stand on involved side
Stabilizing Hand: posterior-lateral side of involved wrist to maintain humeral external rotation and downward rotation of the scapula
Action Hand: Medial side of involved elbow

Client Position:
Supine, slide to edge of the table to allow scapula to retract off the side of the table
Flex elbow 90 degrees
Abduct humerus 20 degrees
Downwardly rotate the scapula

Applied Force:
Abduct humerus to upwardly rotate the scapula

7. Micro Order 7, AMC&S Test

Infraspinatus Superior Fibers

Tester Position:
Body: Stand on involved side
Stabilizing Hand: posterior-lateral side of involved shoulder
Action Hand: Around involved wrist
Brace involved elbow on thigh*

Client Position:
Supine, slide to edge of the table
Flex elbow 90 degrees
Adduct humerus 120 degrees
Fully externally rotate humerus

Applied Force:
Internally rotate humerus

Infraspinatus Superior-Middle Fibers

Tester Position:
Body: Stand on involved side
Stabilizing Hand: posterior-lateral side of involved shoulder
Action Hand: Around involved wrist
Brace involved elbow on thigh*

Client Position:
Supine, slide to edge of the table
Flex elbow 90 degrees
Adduct humerus 90 degrees
Fully externally rotate humerus

Applied Force:
Internally rotate humerus

Infraspinatus Inferior-Middle Fibers

Tester Position:
Body: Stand on involved side
Stabilizing Hand: posterior-lateral side of involved shoulder
Action Hand: Around involved wrist
Brace involved elbow on thigh*

Client Position:
Supine, slide to edge of the table
Flex elbow 90 degrees
Abduct humerus 45 degrees
Fully externally rotate humerus

Applied Force:
Internally rotate humerus

Infraspinatus Inferior Fibers

Tester Position:
Body: Stand on involved side
Stabilizing Hand: posterior-lateral side of involved shoulder
Action Hand: Around involved wrist
Brace involved elbow on thigh*

Client Position:
Supine, slide to edge of the table
Flex elbow 90 degrees
Abduct humerus 30 degrees
Fully externally rotate humerus

Applied Force:
Internally rotate humerus

Teres Minor

Tester Position:
Body: Stand on involved side
Stabilizing Hand: posterior-lateral side of involved shoulder
Action Hand: Around involved wrist
Brace involved elbow on thigh*

Client Position:
Supine, slide to edge of the table
Flex elbow 90 degrees
Adduct humerus
Fully externally rotate humerus

Applied Force:
Internally rotate humerus

8. Micro Order 8, AMC&S Test

Intertransversarii

Tester Position:
Body: Stand on same side of muscle being tested**
Grab anterior aspect of both arms to assist in extension and opposite rotation
Stabilizing Hand: Maintain end range extension and rotation by holding uninvolved side shoulder up and back
Action Hand: move hand to posterior-inferior thorax on uninvolved side
Leg across involved thigh to stabilize pelvis

Client Position:
Prone, hands behind head
Fully extend and rotate thorax to opposite side

Applied Force:
Flexion

Interspinalis Lumborum

Tester Position:
Body: Stand on same side of muscle being tested
Grab anterior aspect of both arms to assist in extension
Stabilizing Hand: Maintain end range extension by holding involved side shoulder up*
Action Hand: move hand to posterior-inferior thorax on involved side
Leg across involved thigh to stabilize pelvis

Client Position:
Prone, hands behind head
Fully extend spine

Applied Force:
Flexion

Spinalis Thoracis

Tester Position:
Body: Stand on uninvolved side
Stabilizing Hand: stabilize uninvolved thorax pressing up and in to inferior rib cage
Action Hand: reach arm under knees, grabbing inferior-lateral side of involved

Client Position:
Supine, legs straight
Legs together, arch spine
Cross arms on chest
Take both legs to involved side to create end range spinal sidebend

Applied Force:
Opposite sidebend
Keep body flat in plane of the table

Spinalis Lumborum

Tester Position:
Body: Stand on uninvolved side
Stabilizing Hand: stabilize uninvolved ilium pressing up and in to inferior iliac crest
Action Hand: reach arm under knees, grabbing inferior-lateral side of involved knee

Client Position:
Supine, legs straight
Legs together, arch spine
Cross arms on chest
Take both legs to involved side to create end range spinal sidebend

Applied Force:
Opposite sidebend
Keep body flat in plane of the table

Rotatores Thoracis

Tester Position:
Body: Stand on uninvolved side
Anchor Leg across uninvolved thigh*
Stabilizing Hand: stabilize under involved side thorax
Action Hand: brace posterior shoulder on uninvolved side

Client Position:
Seated with lower legs off table
Flex trunk 90 degrees
Cross arms
Extend through thoracic spine
Slightly rotate to opposite side

Applied Force:
Flexion

Rotatores Lumborum

Tester Position:
Body: Stand on uninvolved side
Anchor leg across uninvolved thigh
Stabilizing Hand: stabilize across involved side ASIS
Action Hand: brace posterior shoulder on uninvolved side

Client Position:
Seated with lower legs off table
Flex trunk 90 degrees
Cross arms
Extend through lumbar spine
Slightly rotate to opposite side

Applied Force:
Flexion

9. Micro Order 9, AMC&S Test

Gluteus Maximus: Iliac

Tester Position:
Body: stand on involved side
Stabilizing hand: anterior, lower ⅓rd of involved thigh
Action hand: posterior, lower ⅓rd of involved thigh**

Client Position:
Prone, 90 degrees of knee flexion
Abduct thigh to tissue tension
Fully extend hip through plane of the thigh

Applied force:
Hip flexion through plane of the thigh

Gluteus Maximus: Sacral

Tester Position:
Body: stand on involved side
Stabilizing hand: anterior, lower ⅓rd of involved thigh
Action hand: posterior, lower ⅓rd of involved thigh**

Client Position:
Prone, 90 degrees of knee flexion
Slight abduction of involved thigh
Fully extend hip

Applied force:
Hip flexion

Gluteus Maximus Coccygeal

Tester Position:
Body: stand on involved side
Stabilizing hand: anterior, lower ⅓rd of involved thigh
Action hand: posterior, lower ⅓rd of involved thigh**

Client Position:
Prone, 90 degrees of knee flexion
Internally rotate involved thigh
Fully extend hip

Applied force:
Hip flexion

10. Micro Order 10, AMC&S Test

Latissimus Dorsi Iliac Fibers

Tester Position:
Body: Stand on involved side
Stabilizing Hand: on posterior scapula
Action Hand: around involved wrist

Client Position:
Prone with elbow extended
Sidebend to involved side
Internally rotate humerus
Extend then adduct the involved humerus

Applied Force:
Abduction of the humerus

Latissimus Dorsi Lumbar Fibers

Tester Position:
Body: Stand on involved side
Stabilizing Hand: on posterior scapula
Action Hand: around involved wrist

Client Position:
Prone with elbow extended
Sidebend to involved side
Internally rotate humerus
Extend the involved humerus

Applied Force:
Flexion of the humerus

Latissimus Dorsi Thoracic Fibers

Tester Position:
Body: Stand on involved side
Stabilizing Hand: on posterior scapula
Action Hand: around involved wrist

Client Position:
Prone with elbow extended
Sidebend to involved side
Internally rotate humerus
Extend and Adduct involved humerus

Applied Force:
Flexion and Abduction of the humerus through the oblique plane

Teres Major Inferior Fibers

Tester Position:
Body: Stand on involved side
Stabilizing Hand: on posterior side of involved scapula
Action Hand: posterior-medial side of involved elbow

Client Position:
Prone with elbow flexed 90 degrees
Place involved hand on ipsilateral PSIS
Extend and horizontally abduct involved humerus

Applied Force:
Flexion and horizontal Adduction of the humerus

Teres Major Superior Fibers

Tester Position:
Body: Stand on involved side
Stabilizing Hand: on posterior side of involved scapula
Action Hand: posterior-medial side of involved elbow

Client Position:
Prone with elbow flexed 120 degrees
Place involved hand on ipsilateral thorax
Extend and horizontally abduct involved humerus

Applied Force:
Flexion and horizontal Adduction of the humerus

Tricep Long Head

Tester Position:
Body: Stand on involved side
Stabilizing Hand: on posterior scapula
Action Hand: around involved wrist

Client Position:
Prone with elbow extended
Palm down
Extend the involved humerus

Applied Force:
Flexion of the humerus

11. Micro Order 11, AMC&S Test

Sub Scapularis Superior Fibers

Tester Position:
Body: Stand on involved side
Stabilizing Hand: anterior-inferior side of involved shoulder
Action Hand: Around involved wrist
Brace involved elbow on thigh*

Client Position:
Supine, slide to edge of the table
Flex elbow 90 degrees
Abduct humerus 120 degrees
Fully internally rotate humerus

Applied Force:
Externally rotate humerus

Sub Scapularis Superior-Middle Fibers

Tester Position:
Body: Stand on involved side
Stabilizing Hand: anterior-inferior side of involved shoulder
Action Hand: Around involved wrist
Brace involved elbow on thigh*

Client Position:
Supine, slide to edge of the table
Flex elbow 90 degrees
Abduct humerus 90 degrees
Fully internally rotate humerus

Applied Force:
Externally rotate humerus

Sub Scapularis Inferior Fibers

Tester Position:
Body: Stand on involved side
Stabilizing Hand: anterior-inferior side of involved shoulder
Action Hand: Around involved wrist
Brace involved elbow on thigh*

Client Position:
Supine, slide to edge of the table
Flex elbow 90 degrees
Abduct humerus 30 degrees
Fully internally rotate humerus

Applied Force:
Externally rotate humerus

12. Micro Order 12, AMC&S Test

Tricep Medial Fibers

Tester Position:
Body: Stand on involved side
Stabilizing Hand: on posterior-medial side of distal humerus
Action Hand: around involved wrist
Brace involved humerus on thigh*

Client Position:
Supine with elbow extended
Abduct humerus 90 degrees
Fully pronate forearm
Fully extend the elbow

Applied Force:
Elbow flexion

Tricep Lateral Head

Tester Position:
Body: Stand on involved side
Stabilizing Hand: on posterior-medial side of distal humerus
Action Hand: around involved wrist
Brace involved humerus on thigh*

Client Position:
Supine with elbow extended
Abduct humerus 90 degrees
Fully supinate forearm
Fully extend the elbow

Applied Force:
Elbow flexion

Articularis Cubiti

Tester Position:
Body: Stand on involved side
Stabilizing Hand: anterior shoulder at AC-joint
Action Hand: around involved wrist

Client Position:
Supine with elbow flexed
Fully pronate forearm

Applied Force:
Elbow flexion

13. Micro Order 13, AMC&S Test

Upper Trapezius Clavicular Fibers

Tester Position:
Body: Stand on involved side
Stabilizing Hand: on underside of AC joint at armpit to maintain upward rotation of the scapula
Action Hand: around involved wrist

Client Position:
Supine with elbow extended
Rotate head to uninvolved side
Fully internally rotate the involved humerus to protract scapula
Abduct humerus then drive upward rotation of the scapula

Applied Force:
Adduction to downwardly rotate the scapula*

Upper Trapezius Scapular Fibers

Tester Position:
Body: Stand on involved side
Stabilizing Hand: on underside of AC joint at armpit to maintain upward rotation of the scapula
Action Hand: around involved wrist

Client Position:
Supine with elbow extended
Rotate head to uninvolved side
Internally rotate involved humerus to protract scapula
Flex and abduct involved humerus 45 degrees
Upwardly rotate the scapula

Applied Force:
Adduction to downwardly rotate the scapula*

Middle Trapezius

Tester Position:
Body: Stand on involved side
Stabilizing Hand: on anterior side of AC joint to maintain retraction of the scapula
Action Hand: around involved wrist

Client Position:
Supine with elbow extended and head in headpiece with scapula off the table
Horizontal Abduction of the humerus to drive retraction of the scapula

Applied Force:
Horizontal Adduction to protract the scapula

Lower Trapezius

Tester Position:
Body: Stand on involved side
Stabilizing Hand: anterior to the AC joint
Action Hand: around involved wrist

Client Position:
Supine with elbow extended and head in headpiece with scapula off the table
Flexion and Horizontal Abduction of the humerus through the oblique plane to drive upward rotation and retraction of the scapula

Applied Force:
Extenson and Horizontal Adduction through the oblique plane to drive protraction and downward rotation of the scapula

Serratus Anterior Upper Fibers

Tester Position:
Body: Stand on involved side
Stabilizing Hand: on posterior-superior humerus to maintain protraction of the scapula
Action Hand: around involved wrist

Client Position:
Supine with elbow extended and palm up
Abduct humerus 90 degrees
Horizontally adduct involved humerus 45 degrees
Lift arm to drive protraction of the scapula

Applied Force:
Horizontal Abduction to retract the scapula

Serratus Anterior Lower Fibers

Tester Position:
Body: Stand on involved side
Stabilizing Hand: on underside of AC joint at armpit to maintain upward rotation of the scapula
Action Hand: around involved wrist

Client Position:
Supine with elbow extended and head in headpiece with scapula off the table
Palm up
Fully flex involved humerus

Applied Force:
Humeral Extension

Subclavius Lateral Fibers

Tester Position:
Body: Stand on involved side
Stabilizing Hand: on underside of AC joint at armpit to maintain upward rotation of the scapula
Action Hand: around involved wrist

Client Position:
Supine with elbow extended
Internally rotate humerus
Flex and fully abduct involved humerus
Upwardly rotate the scapula

Applied Force:
Adduction to downwardly rotate the scapula

Subclavius Medial Fibers

Tester Position:
Body: Stand on involved side
Stabilizing Hand: on underside of AC joint at armpit to maintain upward rotation of the scapula
Action Hand: around involved wrist

Client Position:
Supine, with elbow extended and palm up
Flex and fully abduct involved humerus
Upwardly rotate the scapula

Applied Force:
Humeral Adduction to downwardly rotate the scapula

14. Micro Order 14, AMC&S Test

Supraspinatus Fossa Division

Tester Position:
Body: Stand on involved side
Stabilizing Hand: Anterior to AC joint
Action Hand: around involved wrist

Client Position:
Supine with elbow extended
Fully internally rotate involved humerus to protract the scapula
Abduct humerus 30 degrees

Applied Force:
Adduct the humerus

Supraspinatus Spinal Division

Tester Position:
Body: Stand on involved side
Stabilizing Hand: Posterior* to the AC-joint
Action Hand: around involved wrist

Client Position:
Supine with elbow extended
Abduct humerus 30 degrees

Applied Force:
Adduct the humerus

Posterior Deltoid Medial Fibers

Tester Position:
Body: Stand on involved side
Stabilizing Hand: around involved wrist
Action Hand: Grab humerus above the elbow

Client Position:
Supine, slide to edge of the table
Flex elbow 90 degrees
Fully internally rotate humerus
Horizontally abduct humerus 20 degrees
Abduct humerus to tissue tension

Applied Force:
Adduct humerus

Posterior Deltoid Lateral Fibers

Tester Position:
Body: Stand on involved side
Stabilizing Hand: around involved wrist
Action Hand: Grab humerus above the elbow

Client Position:
Supine, slide to edge of the table
Flex elbow 90 degrees
Fully internally rotate humerus
Abduct humerus to tissue tension

Applied Force:
Adduct humerus

Middle Deltoid Posterior Fibers

Tester Position:
Body: Stand on involved side
Stabilizing Hand: around involved wrist
Action Hand: Grab humerus above the elbow

Client Position:
Supine, slide to edge of the table
Flex elbow 90 degrees
Slightly internally rotate humerus
Abduct humerus to tissue tension

Applied Force:
Adduct humerus

Middle Deltoid Anterior Fibers

Tester Position:
Body: Stand on involved side
Stabilizing Hand: around involved wrist
Action Hand: Grab humerus above the elbow

Client Position:
Supine, slide to edge of the table
Flex elbow 90 degrees
Slightly externally rotate humerus
Abduct humerus to tissue tension

Applied Force:
Adduct humerus

Anterior Deltoid Scapular Fibers

Tester Position:
Body: Stand on involved side
Stabilizing Hand: around involved wrist
Action Hand: Grab humerus above the elbow

Client Position:
Supine, slide to edge of the table
Flex elbow 90 degrees
Internally rotate humerus
Flex and abduct humerus toward ear

Applied Force:
Extend and Adduct humerus

Anterior Deltoid Clavicular Fibers

Tester Position:
Body: Stand on involved side
Stabilizing Hand: around involved wrist
Action Hand: Grab humerus above the elbow

Client Position:
Supine, slide to edge of the table
Flex elbow 90 degrees
Externally rotate humerus
Flex and abduct humerus toward ear

Applied Force:
Extend and Adduct humerus

15. Micro Order 15, AMC&S Test

Pectoralis Minor Inferior Division

Tester Position:
Body: Stand on involved side
Stabilizing Hand: Posterior to AC-joint to maintain protraction and downward rotation of the scapula
Action Hand: around involved wrist

Client Position:
Supine with elbow extended
Externally rotate humerus
Flex humerus 90 degrees
Depress, then protract the scapula through oblique plane

Applied Force:
Flexion and horizontal abduction through the oblique plane to retract and posteriorly rotate the scapula

Pectoralis Minor Superior Division

Tester Position:
Body: Stand on involved side
Stabilizing Hand: Posterior to AC-joint to maintain protraction and downward rotation of the scapula
Action Hand: around involved wrist

Client Position:
Supine with elbow extended
Externally rotate humerus
Flex humerus 90 degrees
Depress, then protract the scapula through horizontal plane

Applied Force:
Horizontal abduction to retract the scapula

16. Micro Order 16, AMC&S Test

Pectoralis Major Sternal Fibers

Tester Position:
Body: Stand on involved side
Stabilizing Hand: on anterior side of distal clavicle to maintain retraction of the scapula
Action Hand: around involved wrist

Client Position:
Supine with elbow extended and head in headpiece with scapula off the table
Internally rotate humerus
Horizontally adduct the humerus
maintain retraction of the scapula

Applied Force:
Horizontal Abduction of the humerus

Pectoralis Major Clavicular Fibers

Tester Position:
Body: Stand on involved side
Stabilizing Hand: on anterior side of distal clavicle to maintain retraction of the scapula
Action Hand: around involved wrist

Client Position:
Supine with elbow extended and head in headpiece with scapula off the table
Internally rotate humerus
Horizontally adduct and flex the humerus through the oblique plane
Maintain retraction of the scapula

Applied Force:
Extend and horizontally abduct the humerus through the oblique plane

Pectoralis Major Costal Division

Tester Position:
Body: Stand on involved side
Stabilizing Hand: on anterior side of distal clavicle to maintain retraction of the scapula
Action Hand: around involved wrist

Client Position:
Supine with elbow extended and head in headpiece with scapula off the table
Internally rotate humerus
Horizontally adduct and extend the humerus through the oblique plane
Maintain retraction of the scapula

Applied Force:
Flex and Horizontally Abduct the humerus through the oblique plane

Biceps Brachii Long Head

Tester Position:
Body: Stand on involved side
Stabilizing Hand: posterior humerus, superior to elbow
Action Hand: around involved wrist

Client Position:
Supine, with head in headrest and scapula off the table
Supinate forearm with elbow slightly flexed
Fully flex humerus

Applied Force:
Extend the humerus

Biceps Brachii Short Head

Tester Position:
Body: Stand on involved side
Stabilizing Hand: anterior shoulder inferior to AC joint
Action Hand: Around involved wrist

Client Position:
Supine, slide to edge of the table
Supinate forearm
Fully flex elbow

Applied Force:
Elbow extension

Coracobrachialis Superior Fibers

Tester Position:
Body: Stand on involved side
Stabilizing Hand: anterior to the AC joint*
Action Hand: around involved wrist

Client Position:
Supine with elbow extended
Palm up
Abduct humerus 30 degrees
Flex humerus 20 degrees

Applied Force:
Extend the humerus

Coracobrachialis Inferior Fibers

Tester Position:
Body: Stand on involved side
Stabilizing Hand: Anterior to the AC-joint**
Action Hand: around involved wrist

Client Position:
Supine with elbow extended
Fully externally rotate humerus
Abduct humerus 30 degrees
Flex humerus 20 degrees

Applied Force:
Extend and abduct the humerus through the oblique plane

17. Micro Order 17, AMC&S Test

Brachialis

Tester Position:
Body: Stand on involved side
Stabilizing Hand: anterior shoulder at AC-joint
Action Hand: Around involved wrist

Client Position:
Supine, slide to edge of the table
Forearm in neutral
Fully flex elbow

Applied Force:
Elbow extension

Brachioradialis Superior Division

Tester Position:
Body: Stand on involved side
Stabilizing Hand: anterior shoulder at AC-joint
Action Hand: Around involved wrist

Client Position:
Supine, slide to edge of the table
Pronate forearm
Fully flex elbow

Applied Force:
Elbow extension

Brachioradialis Inferior Division

Tester Position:
Body: Stand on involved side
Stabilizing Hand: anterior shoulder at AC-joint
Action Hand: Around involved wrist

Client Position:
Supine, slide to edge of the table
Supinate forearm
Flex elbow 90 degrees

Applied Force:
Elbow extension

18. Micro Order18, AMC&S Test

Adductor Magnus Oblique Fibers

Tester Position:
Body: stand at the base of the client on involved side
Stabilizing Hand: opposite ankle
Action Hand: grab involved ankle from above
**Do not force external rotation

Client Position:
Supine, legs straight
Hands behind head
Fully externally rotate involved femur
Move both legs to create end-range adduction on involved side
**Watch for pelvic hike

Applied Force:
Abduction
**maintain knee extension

Adductor Magnus Vertical Fibers

Tester Position:
Body: stand at the base of table
Stabilizing Hand: opposite ankle
Action Hand: grab involved ankle from below

Client Position:
Supine, legs straight
Hands behind head
Move both legs to create end-range adduction on involved side
**Watch for pelvic hike

Applied Force:
Abduction
**maintain knee extension

Adductor Longus Superior

Tester Position:
Body: stand at the base of the table
Stabilizing Hand: opposite ankle
Action Hand: grab involved ankle from below

Client Position:
Supine, legs straight
Hands across chest
Fully externally rotate involved femur
Flex hips: Femur 10 degrees from table
Move both legs to create end-range adduction on involved side
**Watch for pelvic hike

Applied Force:
Abduction
**maintain knee extension

Adductor Longus Inferior

Tester Position:
Body: stand at the base of the table
Stabilizing Hand: opposite ankle
Action Hand: grab involved ankle from below

Client Position:
Supine, legs straight
Hands across chest
Flex hips: Femur 10 degrees from table
Move both legs to create end-range adduction on involved side

Applied Force:
Abduction
**maintain knee extension

Adductor Brevis

Tester Position:
Body: stand at the base of the table
Stabilizing Hand: opposite ankle
Action Hand: grab involved ankle from below

Client Position:
Supine, legs straight
Hands across chest
Fully internally rotate involved femur
Flex involved hip: Femur 10 degrees from table
Fully adduct the femur at the hip while maintaining internal rotation
**Watch for pelvic rotation

Applied Force:
Abduction
**maintain knee extension

Pectineus

Tester Position:
Body: stand at the base of the client
Stabilizing Hand: opposite ankle
Action Hand: grab involved ankle

Client Position:
Supine, legs straight
Hands across chest
Fully externally rotate involved femur
Flex the involved hip 30 degrees
Adduct to midline while maintaining external rotation

Applied Force:
Extension and Abduction through the oblique plane
**maintain knee extension

Gracilis

Tester Position:
Body: stand at the base of the client on involved side
Stabilizing Hand: opposite ankle
Action Hand: grab involved ankle from below

Client Position:
Supine, legs straight
Hands behind head
Fully internally rotate involved femur
Move both legs to create end-range adduction on involved side

Applied Force:
Abduction
**maintain knee extension

19. Micro Order 19, AMC&S Test

Gluteus Medius Anterior Fibers

Tester Position:
Body: stand at the base of the client on involved side
Stabilizing Hand: opposite ASIS or thigh
Action Hand: grab involved ankle

Client Position:
Supine, legs straight
Hands behind head
Fully internally rotate involved femur
Fully abduct the femur at the hip while maintaining internal rotation

Applied Force:
Adduction
**Maintain knee extension

Gluteus Medius Posterior Fibers

Tester Position:
Body: stand at the base of the client on involved side
Stabilizing Hand: opposite ASIS or thigh
Action Hand: grab involved ankle

Client Position:
Supine, legs straight
Hands behind the head
Fully externally rotate involved femur
Fully abduct the femur at the hip while maintaining external rotation

Applied Force:
Adduction
**Maintain knee extension

Gluteus Medius Middle Fibers

Tester Position:
Body: stand at the base of the client on involved side
Stabilizing Hand: opposite ASIS or thigh
Action Hand: grab involved ankle

Client Position:
Supine, legs straight
Hands behind the head
Fully abduct the femur at the hip

Applied Force:
Adduction
**Maintain knee extension

Gluteus Minimus Anterior Fibers

Tester Position:
Body: stand at the base of the client on involved side
Stabilizing Hand: opposite ASIS or thigh
Action Hand: grab involved ankle

Client Position:
Supine, legs straight
Hands behind the head
Fully internally rotate involved femur
Flex hip 25 degrees
Fully abduct the femur at the hip while maintaining internal rotation

Applied Force:
Adduction
**Maintain knee extension

Gluteus Minimus Lateral Fibers

Tester Position:
Body: stand at the base of the client on involved side
Stabilizing Hand: opposite ASIS or thigh
Action Hand: grab involved ankle

Client Position:
Supine, legs straight
Hands behind the head
Fully externally rotate involved femur
Flex hip 25 degrees
Fully abduct the femur at the hip while maintaining external rotation

Applied Force:
Adduction
**Maintain knee extension

20. Micro Order 20, AMC&S Test

Rectus Femoris Straight Head

Tester Position:
Body: Stand on involved side
Stabilizing Hand: Brace involved thigh
Action Hand: grab lower leg at ankle

Client Position:
Supine, hands behind head
Fully flex hip with end range knee extension

Applied Force:
Hip Extension

Rectus Femoris Reflected Head

Tester Position:
Body: Stand on involved side
Stabilizing Hand: Brace involved thigh
Action Hand: grab lower leg at ankle

Client Position:
Supine, hands behind head
Internally rotate involved leg
Fully flex hip with end range knee extension

Applied Force:
Hip Extension

Vastus Intermedius: Medial

Tester Position:
Body: stand on involved side
Stabilizing Hand: posterior thigh behind knee joint
Action Hand: anterior tibia

Client Position:
Supine, hands behind head
Flex involved hip to tissue tension
Fully internally rotate involved tibia
Tibia parallel to table

Applied Force:
knee flexion

Vastus Intermedius: Lateral

Tester Position:
Body: stand on involved side
Stabilizing Hand: posterior thigh behind knee joint
Action Hand: anterior tibia

Client Position:
Supine, hands behind the head
Flex involved hip to tissue tension
Fully externally rotate involved tibia
Tibia parallel to table

Applied Force:
knee flexion

Vastus Medialis: Upper

Tester Position:
Body: stand on involved side
Stabilizing Hand: lateral side of involved knee
Action Hand: grab involved calcaneus

Client Position:
Supine, hands behind head
Flex hip 60° and flex knee 110° so that heel is at level of opposite knee
Fully externally rotate femur with slight abduction
Internally rotate tibia and plantarflex foot

Applied Force:
Internal rotation of femur at hip

Vastus Medialis: Middle

Tester Position:
Body: stand on involved side
Stabilizing Hand: lateral side of involved knee
Action Hand: grab involved calcaneus

Client Position:
Supine, hands behind head
Flex hip 45° and flex knee 75° so that heel is at opposite mid shin level
Fully externally rotate femur with slight abduction
Internally rotate tibia and plantarflex foot

Applied Force:
Internal rotation of femur at hip

Vastus Medialis: Lower

Tester Position:
Body: stand on involved side
Stabilizing Hand: lateral side of involved knee
Action Hand: grab involved calcaneus

Client Position:
Supine, hands behind head
Flex hip 20° and flex knee 20° so that heel is at level of opposite ankle
Fully externally rotate femur with slight abduction
Internally rotate tibia and plantarflex foot

Applied Force:
Internal rotation of femur at hip

Vastus Lateralis: Upper

Tester Position:
Body: stand on uninvolved side
Stabilizing Hand: medial side of involved knee
Action Hand: grab involved calcaneus

Client Position:
Supine, hands behind head
Flex hip 60° and flex knee 110° so that heel is at level of opposite knee
Fully internally rotate femur with slight adduction
Externally rotate tibia and plantarflex foot

Applied Force:
External rotation of femur at hip

Vastus Lateralis: Middle

Tester Position:
Body: stand on uninvolved side
Stabilizing Hand: medial side of involved knee
Action Hand: grab involved calcaneus

Client Position:
Supine, hands behind head
Flex hip 25° and flex knee 75° so that heel is at opposite mid shin level
Fully internally rotate femur with slight adduction
Externally rotate tibia and plantarflex foot

Applied Force:
External rotation of femur at hip

Vastus Lateralis: Lower

Tester Position:
Body: stand on uninvolved side
Stabilizing Hand: medial side of involved knee
Action Hand: grab involved calcaneus

Client Position:
Supine, hands behind head
Flex hip 20° and flex knee 20° so that heel is at level of opposite ankle
Fully internally rotate femur with slight adduction
Externally rotate tibia and plantarflex foot

Applied Force:
External rotation of femur at hip

Articularis Genu

Tester Position:
Body: stand on involved side
Stabilizing Hand: posterior side of involved knee
Action Hand: grab distal, anterior aspect of involved tibia

Client Position:
Supine, hands behind head
Flex hip 20° and flex knee 20°

Applied Force:
Hip extension while maintaining knee flexion

21. Micro Order 21, AMC&S Test

Semitendinosus

Tester Position:
Body: stand on involved side
Stabilizing Hand: grab involved midfoot on medial side
Action Hand: posterior calcaneus on involved side

Client Position:
Supine, with hands behind head
Flex involved hip 80°
Slightly internally rotate and adduct femur
Dorsiflex and fully internally rotate the foot
Full knee flexion

Applied Force:
knee extension

Semimembranosus Lateral

Tester Position:
Body: stand at base of table
Stabilizing Hand: grab involved midfoot on medial side
Action Hand: posterior calcaneus on involved side

Client Position:
Supine, with hands behind head
Flex involved hip 45°
Slightly externally rotate and abduct femur
Dorsiflex and fully internally rotate the foot
Flex knee 90°

Applied Force:
knee extension

Semimembranosus Medial

Tester Position:
Body: stand at base of table
Stabilizing Hand: grab involved midfoot on medial side
Action Hand: posterior calcaneus on involved side

Client Position:
Supine, with hands behind head
Flex involved hip 45°
Slightly internally rotate and adduct femur
Dorsiflex and fully internally rotate the foot
Flex knee 90°

Applied Force:
Knee extension

Bicep Femoris: Short head

Tester Position:
Body: stand on involved side
Stabilizing Hand: grab involved midfoot on medial side
Action Hand: posterior calcaneus on involved side

Client Position:
Supine, with hands behind head
Flex hip 80°
Slightly internally rotate and adduct femur
Dorsiflex and fully externally rotate the foot
Full knee flexion

Applied Force:
knee extension

Bicep Femoris: Long head Fibular

Tester Position:
Body: stand at base of table
Stabilizing Hand: grab involved midfoot on medial side
Action Hand: posterior calcaneus on involved side

Client Position:
Supine, with hands behind head
Flex involved hip 45°
Slightly internally rotate and adduct femur
Dorsiflex and fully externally rotate foot
Flex knee 90°

Applied Force:
knee extension

Bicep Femoris: Long head Tibial

Tester Position:
Body: stand at base of table
Stabilizing Hand: grab involved midfoot on medial side
Action Hand: posterior calcaneus on involved side

Client Position:
Supine, with hands behind head
Flex involved hip 45°
Dorsiflex and fully externally rotate foot
Flex knee 90°

Applied Force:
knee extension

Sartorius

Tester Position:
Body: stand on involved side
Stabilizing Hand: involved foot
Action Hand: Posterior aspect of distal tibia

Client Position:
Supine, with hands behind head
Flex knee 120 degrees
Flex, abduct and externally rotate femur at hip
Place ankle just above uninvolved knee
Internally rotate tibia

Applied Force:
Knee extension while maintaining abducted/externally rotated position

Popliteus

Tester Position:
Body: stand at base of table
Stabilizing Hand: grab calcaneus
Action Hand: medial forefoot

Client Position:
Supine
Flex involved hip and knee 45°
Internally rotate tibia through foot

Applied Force:
External rotation of the tibia through the foot

22. Micro Order 22, AMC&S Test

Posterior Tibialis Fibular Division

Client Position:
Supine, flex hip 45°, flex knee 90°
Plantarflexion and inversion of the foot.
Full adduction of the foot

Tester Position:
Body: stand at the base of the client
Stabilizing Hand: base of heel
Action Hand: medial aspect of the forefoot

Applied Force:
abduction of the foot

Posterior Tibialis Tibial Division

Client Position:
Supine, flex hip 45°, flex knee 90°
Plantarflexion of the foot.
Full adduction of the foot

Tester Position:
Body: stand at the base of the client
Stabilizing Hand: base of heel
Action Hand: medial aspect of the forefoot

Applied Force:
abduction of the foot

23. Micro Order 23, AMC&S Test

Medial Soleus

Client Position:
prone, knee flexed to 90°
adduction of the foot
full plantarflexion of the foot (point foot up and in)

Tester Position:
Body: stand on side of leg being tested
Stabilizing Hand: back of calcaneus
Action Hand: ball of foot (1st met head)

Applied Force:
dorsiflexion of the ankle about the talo-crual joint axis

Lateral Soleus

Client Position:
prone, knee flexed to 90°
Abduction of the foot
full plantarflexion of the foot (point foot up and out)

Tester Position:
Body: stand on side of leg being tested
Stabilizing Hand: back of calcaneus
Action Hand: ball of foot (4th and 5th met heads)

Applied Force:
dorsiflexion of the ankle about talo-crual joint axis

Lateral Gastroc

Client Position:
supine, flex hip 45°, flex knee 90°
External rotation of the foot
full ankle plantarflexion (point foot down and out)

Tester Position:
Body: stand at the base of the client
Stabilizing Hand: anterior, superior aspect of the forefoot
Action Hand: back of the calcaneus

Applied Force:
Knee extension

Medial Gastroc

Client Position:
supine, flex hip 45°, flex knee 90°
Internal rotation of the foot
full ankle plantarflexion (point foot down and in)

Tester Position:
Body: stand at the base of the client
Stabilizing Hand: anterior, superior aspect of the forefoot
Action Hand: back of the calcaneus

Applied Force:
Knee extension

Plantaris

Client Position:
supine, flex hip 45°, flex knee 90°
full ankle plantarflexion (point foot down)

Tester Position:
Body: stand at the base of the client
Stabilizing Hand: anterior, superior aspect of the forefoot
Action Hand: back of the calcaneus

Applied Force:
Knee extension

24. Micro Order 24, AMC&S Test

Anterior Tibialis Tibial Division

Client Position:
Supine, flex hip 45°, flex knee 90°
Adduction and inversion of the foot
Full dorsiflexion of the foot about the talocrural joint axis (through the 1st met)

Tester Position:
Body: stand at the base of the client
Stabilizing Hand: base of heel
Action Hand: Superior, medial aspect of the forefoot

Applied Force:
Plantarflexion of the foot about the talo-crural joint axis

Anterior Tibialis Interosseous Division

Client Position:
Supine, flex hip 45°, flex knee 90°
Inversion of the foot
Full dorsiflexion of the foot about the talocrural joint axis (through the 1st met)

Tester Position:
Body: stand at the base of the client
Stabilizing Hand: base of heel
Action Hand: Superior, medial aspect of the forefoot

Applied Force:
Plantarflexion of the foot about the talo-crural joint axis

25. Micro Order 25, AMC&S Test

Peroneus Brevis Lateral division

Client Position:
Supine, flex hip 45°, flex knee 90°
Plantarflexion, abduction and eversion of the foot

Tester Position:
Body: stand at the base of the client
Stabilizing Hand: base of heel
Action Hand: lateral aspect of the forefoot

Applied Force:
Adduction of the foot

Peroneus Brevis Posterior Division

Client Position:
supine, flex hip 45°, flex knee 90°
Plantarflexion and abduction of the foot
Slight eversion of the foot

Tester Position:
Body: stand at the base of the client
Stabilizing Hand: base of heel
Action Hand: lateral aspect of the forefoot

Applied Force:
Adduction through the plane of foot

26. Micro Order 26, AMC&S Test

Peroneus Tertius Lateral Division

Client Position:
Supine, flex hip 45°, flex knee 90°,
Abduction & eversion of the foot
Full dorsiflexion of the foot about talo-crural joint axis

Tester Position:
Body: stand at the base of the client
Stabilizing Hand: base of heel
Action Hand: Superior, lateral aspect of the forefoot

Applied Force:
Plantarflexion of the foot about the talo-crural joint axis

Peroneus Tertius Anterior Division

Client Position:
Supine, flex hip 45°, flex knee 90°
Eversion of the foot
Full dorsiflexion of the foot about the talo-crural joint axis

Tester Position:
Body: stand at the base of the client
Stabilizing Hand: base of heel
Action Hand: Superior, lateral aspect of the forefoot

Applied Force:
Plantarflexion of the foot about the talo-crural joint axis

27. Micro Order 27, AMC&S Test

Peroneus Longus Metatarsal Division

Client Position:
supine, flex hip 45°, flex knee 90°
Plantarflexion, abduction and eversion of the foot.
Full plantarflexion and eversion of the 1st met (point foot down and out)

Tester Position:
Body: stand at the side of the client's involved foot
Stabilizing Hand: base of heel
Action Hand: Under 1st met head

Applied Force:
Dorsiflexion and inversion of the foot (up and in) through the 1st metatarsal head

Peroneus Longus Cuneiform Division

Client Position:
supine, flex hip 45°, flex knee 90°
Plantarflexion and eversion of the foot
Full abduction of the foot

Tester Position:
Body: stand at the side of the client's involved foot
Stabilizing Hand: base of heel
Action Hand: lateral aspect of the forefoot

Applied Force:
adduction of the foot

28. Micro Order 28, AMC&S Test

Extensor Hallucis Longus Fibular Division

Client Position:
Supine, leg straight
Dorsiflexion with inversion of the foot.
Extension of the distal phalanx of hallux (driving ankle dorsiflexion)

Tester Position:
Body: Stand at base of the client
Stabilizing Hand: Brace proximal phalanx of hallux
Action Hand: brace dorsal surface of distal phalanx

Applied Force:
Plantarflexion of distal phalanx of hallux
**do not force dorsiflexion of proximal phalanx

Extensor Hallucis Longus Interosseous Division

Client Position:
Supine, leg straight
Dorsiflexion of the foot
Extension of the distal phalanx of hallux (driving ankle dorsiflexion)

Tester Position:
Body: Stand at base of the client
Stabilizing Hand: Brace proximal phalanx of hallux
Action Hand: brace dorsal surface of distal phalanx

Applied Force:
Plantarflexion of distal phalanx of hallux
**do not force dorsiflexion of proximal phalanx

Extensor Hallucis Brevis

Client Position:
Supine, leg straight
Dorsiflexion of the foot
Dorsiflexion of proximal phalanx of the hallux (driving ankle dorsiflexion)

Tester Position:
Body: Stand at base of the client
Stabilizing Hand: Brace 1st metatarsal head
Action Hand: brace proximal phalanx of hallux

Applied Force:
Plantarflexion of the proximal phalanx of the hallux 29. Micro Order 29, AMC&S Test

Extensor Digitorum Longus Lateral Division

Client Position:
Supine, leg straight
Dorsiflexion with eversion of the foot.
Extension of the middle and distal phalanges of lateral two toes (driving ankle dorsiflexion)

Tester Position:
Body: Stand at base of the client
Stabilizing Hand: Brace proximal phalanges of lateral 2 toes
Action Hand: brace dorsal surface of middle and distal phalanges of lateral 2 toes

Applied Force:
Simultaneous plantarflexion of the middle and distal phalanges of lateral 2 toes
**do not force dorsiflexion of proximal phalanx

Extensor Digitorum Longus Medial Division

Client Position:
Supine, leg straight
Dorsiflexion with eversion of the foot.
Extension of the middle and distal phalanges of medial two toes (driving ankle dorsiflexion)

Tester Position:
Body: Stand at base of the client
Stabilizing Hand: Brace proximal phalanges of medial 2 toes
Action Hand: brace dorsal surface of middle and distal phalanges of medial 2 toes

Applied Force:
Simultaneous plantarflexion of middle and distal phalanges of the medial 2 toes
**do not force dorsiflexion of proximal phalanx

Extensor Digitorum Brevis

Client Position:
Supine, leg straight
Dorsiflexion of foot
Dorsiflex proximal phalanx of 2nd, 3rd & 4th toes (driving ankle dorsiflexion)

Tester Position:
Body: Stand distal to body
Stabilizing Hand: Brace plantar surface of foot
Action Hand: brace dorsal surface of proximal phalanx of the middle 3 toes

Applied Force:
Plantarflexion of the proximal phalanx of the middle 3 toes

1st Dorsal Interossei

Client Position:
Supine, leg straight
Foot Neutral
Extend and abduct the proximal phalanx of the 2nd toe

Tester Position:
Body: Stand distal to body
Stabilizing Hand: Brace 2nd metatarsal
Action Hand: brace medial aspect of proximal phalanx of 2nd toe

Applied Force:
Adduction of the proximal phalanx of the 2nd toe toward the midline of foot*

2nd Dorsal Interossei

Client Position:
Supine, leg straight
Foot Neutral
Extend and abduct the proximal phalanx of the 2nd toe

Tester Position:
Body: Stand distal to body
Stabilizing Hand: Brace 2nd metatarsal
Action Hand: brace lateral aspect of proximal phalanx of 2nd toe

Applied Force:
Adduction of the proximal phalanx of 2nd toe toward the midline of the foot*

3rd Dorsal Interossei

Client Position:
Supine, leg straight
Foot neutral
Extend and abduct the proximal phalanx of the 3rd toe

Tester Position:
Body: Stand distal to body
Stabilizing Hand: Brace 3rd metatarsal
Action Hand: brace lateral aspect of proximal phalanx of 3rd toe

Applied Force:
Adduction of the proximal phalanx of the 3rd toe toward the midline of the foot

4th Dorsal Interossei

Client Position:
Supine, leg straight
Foot Neutral
Extend and abduct the proximal phalanx of the 4th toe

Tester Position:
Body: Stand distal to body
Stabilizing Hand: Brace 4th metatarsal
Action Hand: brace lateral aspect of proximal phalanx of 4th toe

Applied Force:
Adduction of the proximal phalanx of the 4th toe toward the midline of the foot

30. Micro Order 30, AMC&S Test

Flexor Hallucis Longus Fibular Division

Client Position:
Supine, leg straight
Plantarflexion and inversion of the foot.
Full Plantarflexion of distal phalanx of the hallux (driving ankle plantarflexion) (Point big toe down and in)

Tester Position:
Body: Stand to the side of involved foot
Stabilizing Hand: Brace proximal phalanx of the hallux
Action Hand: brace plantar surface of distal phalanx of the hallux

Applied Force:
Dorsiflexion of the distal phalanx of the hallux
**do not force plantarflexion of proximal phalanx

Flexor Hallucis Longus Interosseous Division

Client Position:
Supine: leg straight
Plantarflexion of the foot
Full Plantarflexion of distal phalanx of the hallux (driving ankle plantarflexion) (point big toe down)

Tester Position:
Body: Stand to the side of involved foot
Stabilizing Hand: Brace the proximal phalanx of the hallux
Action Hand: brace plantar surface of distal phalanx of the hallux

Applied Force:
Dorsiflexion of the distal phalanx of the hallux
**do not force plantarflexion of proximal phalanx

Flexor Hallucis Brevis 1st Cuneiform Division

Client Position:
Supine, straight leg
Plantarflexion of the foot
Plantarflexion and Inversion of the proximal phalanx of the hallux (relative to midline of the foot)

Tester Position:
Body: Stand to the side of involved foot
Stabilizing Hand: Brace 1st metatarsal
Action Hand: brace the inferior, lateral aspect of the proximal phalanx of the hallux

Applied Force:
Dorsiflexion and Eversion of the proximal phalanx of the hallux (relative to midline of the foot)

Flexor Hallucis Brevis Cuboid Division

Client Position:
Supine, straight leg
Plantarflexion of the foot
Plantarflexion and Eversion of the proximal phalanx of the hallux (relative to midline of the foot)

Tester Position:
Body: Stand to the side of the involved foot
Stabilizing Hand: Brace the 1st metatarsal
Action Hand: brace the inferior, medial aspect of the proximal phalanx of the hallux

Applied Force:
Dorsiflexion and Inversion of the proximal phalanx of the hallux (relative to midline of the foot)

Flexor Hallucis Brevis 3rd Cuneiform Division

Client Position:
Supine
Plantarflexion of the foot
Plantarflexion of the proximal phalanx of the hallux

Tester Position:
Body: Stand to the side of involved foot
Stabilizing Hand: Brace 1st metatarsal
Action Hand: brace proximal phalanx of hallux

Applied Force:
Dorsiflexion of the proximal phalanx of the hallux

Adductor Hallucis Oblique Head

Client Position:
Supine, leg straight
Plantarflexion of the foot
Plantarflexion and adduction of the proximal phalanx of the hallux

Tester Position:
Body: Stand to the side of involved foot
Stabilizing Hand: Brace 1st met
Action Hand: brace proximal phalanx of the hallux

Applied Force:
Abduction of the proximal phalanx of hallux

Adductor Hallucis Transverse Head: Lateral Division

Client Position:
Supine, leg straight
Plantarflexion of the foot
Plantarflexion and inversion of the 4th,and 5th mets with plantarflexion at the 1st MTP-joint

Tester Position:
Body: Stand distal to body
Stabilizing Hand: brace the 1st met head
Action Hand: Brace dorsum of 4th and 5th metatarsal heads

Applied Force:
Dorsiflexion and eversion of the 4th and 5th metatarsals

Adductor Hallucis Transverse Head: Lateral Division

Client Position:
Supine, leg straight
Plantarflexion of the foot
Plantarflexion and inversion of the 4th,and 5th mets with plantarflexion at the 1st MTP-joint

Tester Position:
Body: Stand distal to body
Stabilizing Hand: brace the 1st met head
Action Hand: Brace dorsum of 4th and 5th metatarsal heads

Applied Force:
Dorsiflexion and eversion of the 4th and 5th metatarsals

Abductor Hallucis Longus Supinator Division

Client Position:
Supine, flex hip 45° and flex knee 90°
Plantarflexion of the foot
invert the forefoot on the rearfoot
Adduct the forefoot on the rearfoot

Tester Position:
Body: Stand distal to body
Stabilizing Hand: Brace the calcaneus
Action Hand: medial aspect of hallux and 1st met

Applied Force:
Abduction of the forefoot on rearfoot through the hallux
Abductor Hallucis Longus Adductor Division

Client Position:
Supine, flex hip 45° and flex knee 90°
Plantarflexion of the foot
Adduct of the forefoot on the rearfoot

Tester Position:
Body: Stand distal to body
Stabilizing Hand: Brace the calcaneus
Action Hand: medial aspect of hallux and 1st met

Applied Force:
Abduction of the forefoot on rearfoot through the hallux

31. Micro Order 31, AMC&S Test

Flexor Digitorum Longus Lateral Division

Client Position:
Supine, leg straight
Plantarflexion with inversion of the foot
Plantarflex distal phalanges of lateral 2 toes (driving plantarflexion of the foot) (point toes down and in)

Tester Position:
Body: Stand at side of involved foot
Stabilizing Hand: Brace middle phalanges
Action Hand: plantar surface of distal phalanges

Applied Force:
Dorsiflex distal phalanges of lateral 2 toes

Flexor Digitorum Longus Medial Division

Client Position:
Supine, leg straight
Plantarflexion with inversion of the foot
Plantarflex distal phalanges of medial 2 toes (driving ankle plantarflexion) (point toes down and in)

Tester Position:
Body: Stand at side of involved foot
Stabilizing Hand: Brace middle phalanges
Action Hand: plantar surface of distal phalanges

Applied Force:
Dorsiflex distal phalanges of medial 2 toes

Flexor Digitorum Brevis Lateral Division

Client Position:
Supine, leg straight
Plantarflexion with inversion of the foot
Plantarflexion of middle phalanx of lateral 2 toes.
Maintain extension of distal phalanx

Tester Position:
Body: Stand at side of involved foot
Stabilizing Hand: Brace proximal phalanx of lateral 2 toes
Action Hand: brace base of middle phalanges

Applied Force:
Dorsiflexion of the middle phalanx of the lateral 2 toes while maintaining extension of the distal phalanx

Flexor Digitorum Brevis Medial Division

Client Position:
Supine, leg straight
Plantarflexion with inversion of the foot
Plantarflexion of the middle phalanx of medial 2 toes.
Maintain extension of distal phalanx

Tester Position:
Body: Stand at side of involved foot
Stabilizing Hand: Brace proximal phalanx of the medial 2 toes
Action Hand: brace base of middle phalanges

Applied Force:
Dorsiflexion of the middle phalanx of medial 2 toes while maintaining extension of the distal phalanx

Quadratus Plantae Lateral Head

Client Position:
Supine, leg straight
Plantarflexion with inversion of the foot
Plantarflex proximal phalanges of 4th and 5th toes while maintaining extension of middle and distal phalanx

Tester Position:
Body: Stand at side of involved foot
Stabilizing Hand: Brace metatarsal heads of lateral 2 toes
Action Hand: brace base of proximal phalanges of lateral 2 toes

Applied Force:
Dorsiflexion of the proximal phalanx of lateral 2 toes while maintaining extension of middle and distal phalanges

Quadratus Plantae Medial Head

Client Position:
Supine, leg straight
Plantarflexion with inversion of the foot
Plantarflexion of the proximal phalanges of 2nd and 3rd toes while maintaining extension of middle and distal phalanges

Tester Position:
Body: Stand at side of involved foot
Stabilizing Hand: Brace metatarsal heads of medial 2 toes
Action Hand: brace base of proximal phalanx of medial 2 toes

Applied Force:
Dorsiflexion of the proximal phalanx of medial 2 toes while maintaining extension of middle and distal phalanges

4th Plantar Lumbrical

Client Position:
Supine, leg straight
Plantarflexion with inversion of the foot*
Extension of the proximal phalanx of the 5th toe while maintaining extension of the middle and distal phalanges

Tester Position:
Body: Stand distal to body
Stabilizing Hand: Brace 5th metatarsal head
Action Hand: brace base of proximal phalanx of 5th metatarsal while maintaining middle and distal extension

Applied Force:
Dorsiflexion of the 5th toe

3rd Plantar Lumbrical

Client Position:
Supine, leg straight
Plantarflexion with inversion of the foot*
Extension of the proximal phalanx of the 4th toe while maintaining extension of the middle and distal phalanges

Tester Position:
Body: Stand distal to body
Stabilizing Hand: Brace 4th metatarsal head
Action Hand: brace base of proximal phalanx of 4th metatarsal while maintaining middle and distal extension

Applied Force:
Dorsiflexion of the 4th toe

2nd Plantar Lumbrical

Client Position:
Supine, leg straight
Plantarflexion with inversion of the foot*
Extension of the proximal phalanx of the 3rd toe while maintaining extension of the middle and distal phalanges

Tester Position:
Body: Stand distal to body
Stabilizing Hand: Brace 3rd metatarsal head
Action Hand: brace base of proximal phalanx of 3rd metatarsal while maintaining middle and distal extension

Applied Force:
Dorsiflexion of the 3rd toe

1st Lumbrical

Client Position:
Supine, leg straight
Plantarflexion with inversion of the foot*
Extension of the proximal phalanx of the 2nd toe while maintaining extension of the middle and distal phalanges

Tester Position:
Body: Stand distal to body
Stabilizing Hand: Brace 2nd metatarsal head
Action Hand: brace base of proximal phalanx of 2nd metatarsal while maintaining middle and distal extension

Applied Force:
Dorsiflexion of the 2nd toe

3rd Plantar Interossei

Client Position:
Supine, leg straight
Plantarflexion with inversion of the foot
Plantarflexion of the proximal phalanx of the 5th toe

Tester Position:
Body: Stand distal to body
Stabilizing Hand: Brace 5th metatarsal
Action Hand: brace medial aspect of proximal phalanx of the 5th toe

Applied Force:
Dorsiflexion of the proximal phalanx of the 5th toe

2nd Plantar Interossei

Client Position:
Supine, leg straight
Plantarflexion with inversion of the foot
Plantarflexion of the proximal phalanx of the 4th toe

Tester Position:
Body: Stand distal to body
Stabilizing Hand: Brace 4th metatarsal
Action Hand: brace medial aspect of proximal phalanx of the 4th toe

Applied Force:
Dorsiflexion of the proximal phalanx of the 4th toe

1st Plantar Interossei

Client Position:
Supine, leg straight
Plantarflexion with inversion of the foot
Plantarflexion of the proximal phalanx of the 3rd toe

Tester Position:
Body: Stand distal to body
Stabilizing Hand: Brace 3rd metatarsal
Action Hand: brace medial aspect of proximal phalanx of the 3rd toe

Applied Force:
Dorsiflexion of the proximal phalanx of 3rd toe

Abductor Digiti Minimi

Client Position:
Supine, hip flexed 45 degrees with 90 degrees of knee flexion
Plantarflexion of the foot.
Abduction of the forefoot on rearfoot

Tester Position:
Body: Stand distal to body
Stabilizing Hand: Brace the calcaneus
Action Hand: Lateral and distal aspect of 5th metatarsal

Applied Force:
Adduction of the forefoot on the rearfoot

Flexor Digiti Minimi Brevis

Client Position:
Supine, leg straight
Plantarflexion of the foot
Flexion and abduction of the proximal phalanx of 5th toe
Keep distal phalanx extended

Tester Position:
Body: Stand distal to body
Stabilizing Hand: Brace the 5th metatarsal
Action Hand: brace base of 5th proximal phalange

Applied Force:
Dorsiflexion and adduction of the proximal phalanx of 5th toe

32. Micro Order 32, AMC&S Test

Longus Capitis

Tester Position:
Body: Stand distal to body
Stabilizing Hand: Brace hand on back of head
Action Hand: brace hand on front of head

Client Position:
Supine, tuck chin to flex head on neck
Rotate 20° toward uninvolved side
Fully flex cervical spine

Applied Force:
extend head on neck through plane of the head

Longus Colli: Superior Oblique Fibers

Tester Position:
Body: Stand distal to body
Stabilizing Hand: Brace hand on contralateral side of head
Action Hand: brace hand on ipsilateral side of head

Client Position:
Supine, fully rotate head to uninvolved side
Extend head on neck (Chin up)
Fully flex neck on trunk

Applied Force:
extend neck on trunk through sagittal plane

Longus Colli: Vertical Fibers

Tester Position:
Body: Stand distal to body
Stabilizing Hand: Brace hand on contralateral side of head
Action Hand: brace hand on ipsilateral side of head

Client Position:
Supine, fully rotate head to uninvolved side
Extend head on neck (Chin up)
Flex neck on trunk 25 degrees

Applied Force:
extend neck on trunk through sagittal plane

Longus Colli: Inferior Oblique Fibers

Tester Position:
Body: Stand distal to body
Stabilizing Hand: Brace hand on contralateral side of head
Action Hand: brace hand on ipsilateral side of head

Client Position:
Supine, fully rotate head to uninvolved side
Extend head on neck (Chin up)
Flex neck on trunk 5 degrees

Applied Force:
extend neck on trunk through sagittal plane

Mylohyoid

Tester Position:
Body: Stand distal to body
Stabilizing Hand: Brace hand on back of head
Action Hand: brace hand on front of head

Client Position:
Supine, tuck chin to flex head on neck
Rotate 20° toward uninvolved side
Clench teeth with tongue to the roof of the mouth
Fully flex cervical spine

Applied Force:
extend head on neck through plane of the head

Sternohyoid

Tester Position:
Body: Stand distal to body
Stabilizing Hand: Brace hand on back of head
Action Hand: brace hand on front of head

Client Position:
Supine, Rotate head 20° toward uninvolved side
Extend head on neck (chin up)
Clench teeth with tongue to the roof of the mouth
Fully flex cervical spine

Applied Force:
Extend neck through plane of the head

Rectus Capitis Anterior

Tester Position:
Body: Stand distal to body
Stabilizing Hand: Brace on back of the head
Action Hand: brace anterior side of head

Client Position:
full flexion of head on neck and neck on trunk
Rotate head 45° to contralateral side

Applied Force:
Extend head on neck through oblique plane

33. Micro Order 33, AMC&S Test

Multifidus Cervicis Inferior Division

Tester Position:
Body: Stand distal to body
Stabilizing Hand: Brace hand around opposite ear
Action Hand: brace around involved ear

Client Position:
Full rotation of neck to opposite side
Lateral Flexion of neck to involved side
Tuck Chin

Applied Force:
Laterally flex neck though plane of table

Multifidus Cervicis Superior Division

Tester Position:
Body: Stand distal to body
Stabilizing Hand: Brace hand around opposite ear
Action Hand: brace around involved ear

Client Position:
Full rotation of neck to opposite side
Lateral Flexion of neck to involved side
Chin Up

Applied Force:
Laterally flex neck through plane of table

Sternocleidomastoid Sternal Fibers

Tester Position:
Body: Stand distal to body
Stabilizing Hand: Brace hand around opposite ear
Action Hand: brace palm of hand over temporal bone

Client Position:
Supine, Extend head on neck
Rotate 45° to contralateral side
Fully flex neck on trunk

Applied Force:
Extend neck on trunk in sagittal plane

Sternocleidomastoid Clavicular Fibers

Tester Position:
Body: Stand distal to body
Stabilizing Hand: Brace hand around opposite ear
Action Hand: brace around involved ear

Client Position:
Supine, Extend head on neck
Rotate 45° to contralateral side
Fully flex neck on trunk

Applied Force:
extend and sidebend neck on trunk though oblique plane

Longissimus Capitis

Tester Position:
Body: Stand on involved side of the body
Stabilizing Hand: Brace hand on contralateral side of head: use elbow to brace thoracic spine
Action Hand: Posterior side of head

Client Position:
Prone, full rotation to involved side
full extension of neck on trunk then head on neck

Applied Force:
Flex head on neck in sagittal plane (Separate mastoid from TP's)

Longissimus Cervicis

Tester Position:
Body: Stand on involved side of the body
Stabilizing Hand: Brace hand on contralateral side of head: use elbow to brace thoracic spine
Action Hand: Posterior side of head

Client Position:
Prone, full rotation to involved side
Full extension of neck on trunk
Flex head on neck

Applied Force:
flex neck on trunk through the sagittal plane (separate TP's)

Splenius Capitis

Tester Position:
Body: Stand on involved side of the body
Stabilizing Hand: Brace uninvolved side of head: use elbow to brace thoracic spine
Action Hand: posterior-lateral side of head

Client Position:
Prone, fully extend cervical spine
Fully rotate to involved side
Extend head on neck

Applied Force:
flex head through Sagittal plane

Splenius Cervicis

Tester Position:
Body: Stand on involved side of body
Stabilizing Hand: Brace uninvolved side of head: use elbow to brace thoracic spine
Action Hand: Posterior-lateral side of head

Client Position:
Prone, fully extend cervical spine
Fully rotate to involved side
Tuck chin

Applied Force:
flex head through sagittal plane

Iliocostalis Cervicis

Tester Position:
Body: Stand on involved side of body
Stabilizing Hand: Brace uninvolved side of head: use elbow to brace thoracic spine
Action Hand: Lateral side of head

Client Position:
Prone, tuck chin
Rotate head to look over involved shoulder

Applied Force:
flex head through oblique plane

Rotatores Cervicis

Tester Position:
Body: Stand distal to body
Stabilizing Hand: Brace hand around opposite ear and skull
Action Hand: brace around involved skull

Client Position:
Full rotation of neck to opposite side
Lateral flexion to involved side
Chin up

Applied Force:
Counter-rotation of head and neck

Rectus Capitis Posterior Major

Tester Position:
Body: Stand distal to body
Stabilizing Hand: Brace hand around opposite side skull
Action Hand: brace around involved skull

Client Position:
Full flexion of neck on trunk
Extend head on neck
Rotate head 45° to ipsilateral side

Applied Force:
Rotate head to contralateral side

Obliques Capitis Inferior

Tester Position:
Body: Stand distal to body
Stabilizing Hand: Brace hand around opposite ear
Action Hand: brace around involved ear

Client Position:
full flexion of head on neck and neck on trunk (chin down)
Full rotation of head to ipsilateral side

Applied Force:
Rotate head to contralateral side

34. Micro Order 34, AMC&S Test

Semispinalis Capitis

Tester Position:
Body: Stand on involved side of body
Stabilizing Hand: anterior side of head: use elbow to stabilize thoracic spine
Action Hand: Brace hand on back of skull

Client Position:
Prone, rotate 20° to uninvolved side
Extend head
Fully extend cervical spine

Applied Force:
flex head and neck through plane of head

Semispinalis Cervicis

Tester Position:
Body: Stand on involved side of the body
Stabilizing Hand: anterior side of head: use elbow to stabilize thoracic spine
Action Hand: Brace hand on back of skull

Client Position:
Prone, rotate 20° to uninvolved side
Fully extend cervical spine
Flex head on neck

Applied Force:
Flex neck on trunk through plane of head

Spinalis Capitis

Tester Position:
Body: Stand on involved side of the body
Stabilizing Hand: Anterior side of skull
Action Hand: Brace hand behind ear on skull

Client Position:
Prone, slightly rotate to involved side
extend cervical spine 20°
extend head on neck

Applied Force:
flex head on trunk in sagittal plane in cocking motion

Spinalis Cervicis

Tester Position:
Body: Stand on side of body
Stabilizing Hand: Brace opposite shoulder
Action Hand: Brace hand behind ear on skull

Client Position:
Prone, slightly rotate to involved side
Flex head on neck
Extend cervical spine 20°

Applied Force:
flex head sagittal plane

Interspinalis Cervicis

Tester Position:
Body: Stand distal to body
Stabilizing Hand: Brace hand around front of head
Action Hand: posterior side of head on involved side

Client Position:
Extension of head and neck

Applied Force:
Cervical flexion

Obliques Capitis Superior

Tester Position:
Body: Stand distal to body
Stabilizing Hand: Brace hand around uninvolved side of skull
Action Hand: brace posterior skull on involved side

Client Position:
Full rotation of head to contralateral side
Flexion of neck on trunk
Flexion of head on neck

Applied Force:
extend head obliquely toward opposite ear

Rectus Capitis Posterior Minor

Tester Position:
Body: Stand distal to body
Stabilizing Hand: Brace hand around uninvolved side of skull
Action Hand: brace around involved side skull

Client Position:
Slight flexion of neck on trunk
Full extension of head on neck (chin up)
Slight rotation to involved side

Applied Force:
Flex head on neck

35. Micro Order 35, AMC&S Test

Posterior Scalenes AMC&S Test

Tester Position:
Body: Stand distal to body
Stabilizing Hand: Brace hand on uninvolved side of head above ear
Action Hand: brace hand on involved side of head above ear

Client Position:
Supine, grab sides of table
rotate head 20° to uninvolved side
Slightly flex cervical spine
Flex and fully side bend neck on thorax through oblique plane
Chin up

Applied Force:
laterally flex and extend neck on thorax through oblique plane

Middle Scalenes

Tester Position:
Body: Stand distal to body
Stabilizing Hand: Brace hand on uninvolved side of head above ear
Action Hand: brace hand on involved side of head above ear

Client Position:
Supine, grab sides of table
rotate head 20° to uninvolved side
Slightly flex cervical spine
Flex and fully side bend neck on thorax through oblique plane
Chin up

Applied Force:
laterally flex neck on trunk through oblique plane with force coming from anterior to the ear

Anterior Scalenes

Tester Position:
Body: Stand distal to body
Stabilizing Hand: Brace hand around opposite side of head
Action Hand: brace hand on involved side of head

Client Position:
Supine, flex cervical spine 45°
rotate 20° away
Fully sidebend neck
extend head on neck (chin up)

Applied Force:
Laterally flex neck on trunk in oblique plane

Anterior Intertransversarii

Tester Position:
Body: Stand distal to body
Stabilizing Hand: Brace hand around opposite side of head
Action Hand: brace around involved side of head

Client Position:
Slight rotation of head to involved side
Lateral flexion of neck to same side
Tuck Chin

Applied Force:
Laterally flex neck coronal plane

Omohyoid

Tester Position:
Body: Stand distal to body
Stabilizing Hand: Brace hand around opposite side of head
Action Hand: brace around involved side of head

Client Position:
Full flexion of neck on trunk
20 degrees of rotation to opposite side
Capital extension
Shrug involved shoulder
Teeth clenched with tongue to roof of mouth

Applied Force:
Laterally flex and extend neck on trunk through oblique plane

Rectus Capitis Lateralis

Tester Position:
Body: Stand distal to body
Stabilizing Hand: Brace hand around uninvolved side of head
Action Hand: brace around involved side of head

Client Position:
Lateral flexion of head on neck

Applied Force:
Laterally flex head on neck through coronal plane

36. Micro Order 36, Muscle Test

Extensor Carpi Radialis Longus: Abductor Division

Client supine
Full Elbow flexion with forearm pronated*
Abduct/Radial Deviate hand at wrist and fully extend wrist emphasizing pressure through the 2nd met

Force: Wrist Flexion with Adduction/Ulnar Deviation (Force through 2nd met)

Extensor Carpi Radialis Longus: Extensor Division Muscle Test

Client supine
Full Elbow flexion with forearm pronated*
Fully extend wrist emphasizing pressure through the 2nd met

Force: Wrist Flexion (Force through 2nd met)

Extensor Carpi Ulnaris: Adductor Division
Client supine
Full Elbow flexion with forearm pronated*
Adduct/Ulnar Deviate hand at wrist and fully extend wrist emphasizing pressure through the 5th met
Force: Wrist Flexion with Adduction/Ulnar Deviation (force through 5th met)

Extensor Carpi Ulnaris: Extensor Division
Client supine
Full Elbow flexion with forearm pronated*
Fully extend wrist emphasizing pressure through the 5th met
FORCE: Wrist Flexion (Force through 5th met)

Extensor Carpi Radialis Brevis
Client supine
Full Elbow flexion with forearm pronated*
Fully extend wrist emphasizing pressure through the 3rd met
FORCE: Wrist Flexion with Adduction/Ulnar Deviation (Force through 3rd met)

37. Micro Order 37, Muscle Test

Flexor Carpi Radialis: Abductor Division
Client supine
90° Elbow flexion with forearm supinated
Abduct/Radial Deviate and fully flex hand at wrist emphasizing pressure through 2nd met

Force: Wrist extension with adduction/ulnar deviation (Force through 2nd met)

Flexor Carpi Radialis: Flexor Division
Client supine
90° Elbow flexion with forearm supinated
Fully flex hand at wrist emphasizing pressure through 2nd met

Force: Wrist extension (Force through 2nd met)

Flexor Carpi Ulnaris: Adductor Division
Client supine
90° Elbow flexion with forearm supinated
Adduct/Ulnar Deviate and fully flex hand at wrist emphasizing pressure through 5th met

Force: Wrist extension with adduction/ulnar deviation (Force through 5th met)

Flexor Carpi Ulnaris: Flexor Division
Client supine
90° Elbow flexion with forearm supinated
Fully flex hand at wrist emphasizing pressure through 5th met

Force: Wrist extension (Force through 5th met)

Palmaris Longus
Client supine
90° Elbow flexion with Supination and full wrist flexion
Point fingers and thumb together (eagles beak)

Force: Wrist extension

38. Micro Order 38, Muscle Test

Anconeus: Ulnar Division
Client supine
Fully extend elbow and supinate forearm

Force:
Pronate forearm, maintaining elbow extension

Anconeus: Olecranon Division
Client supine,
Flex elbow 45° and supinate forearm

Force:
Pronate forearm, with elbow flexed 45°

Supinator: Olecranon Division Muscle Test
Client supine
Supinate forearm with 90 degrees of elbow flexion

Force: Pronate forearm

Supinator: Ulnar Division
Client supine
Supinate forearm with full elbow flexion

Force: Pronate forearm

39. Micro Order 39, Muscle Test

Pronator Teres: Humeral Division
Client supine
Elbow extension with full pronation of forearm

Force: Supination of forearm

Pronator Teres: Ulnar Division
Client supine
Flex elbow 45° with full pronation of forearm

Force: Supination of forearm

Pronator Quadratus: Proximal Division
Client supine
Elbow flexed 90° with full pronation of the forearm (hand straight)

Force: Supination of forearm

Pronator Quadratus: Distal Division
Client supine
Full flexion of the elbow and full pronation of the forearm

Force: Supination of Forearm

40. Micro Order 40, Muscle Test

Extensor Pollicis Longus: Ulnar Division
Client supine
Elbow flexed 90 Degrees with forearm supinated
Extend distal phalanx of thumb with metacarpal abducted and radial deviation of the hand at the wrist
Brace proximal phalanx

Force: Flex distal phalanx on proximal phalanx

Extensor Pollicis Longus: Septal Division Muscle Test
Client supine
Elbow flexed 90 Degrees forearm pronated
Extend distal phalanx of thumb with metacarpal abducted and radial deviation of the hand at the wrist
Brace proximal phalanx

Force: Flex distal phalanx on proximal phalanx

Extensor Pollicis Brevis: Radial Division
Client supine
Elbow flexed 90 Degrees with forearm supinated
Extend and abduct proximal phalanx while flexing distal phalanx of thumb
Radial deviation of the hand at the wrist
Brace 1st metacarpal

Force: Flex and adduct proximal phalanx of the thumb

Extensor Pollicis Brevis: Septal Division
Client supine
Elbow flexed 90 Degrees with forearm pronated
Extend and abduct proximal phalanx while flexing distal phalanx of thumb
Radial deviation of the hand at the wrist
Brace 1st metacarpal

Force: Flex and adduct proximal phalanx of the thumb

Abductor Pollicis Longus: Radial Division
Client supine
Elbow flexed 90 degrees with forearm supinated
Abduct and extend 1st metacarpal (maintain flexion of distal phalanx)
Radial deviation of the hand at the wrist
Brace trapezium

Force: Flex and Adduct 1st metacarpal

Abductor Pollicis LonguS: Ulnar Division
Client supine
Elbow flexed 90 degrees with forearm pronated
Abduct and extend 1st metacarpal (maintain flexion of distal phalanx)
Radial deviation of the hand at the wrist
Brace trapezium

Force: Flex and Adduct 1st metacarpal

41. Micro Order 41, Muscle Test

Flexor Pollicis Longus
Client supine
Elbow flexed 90 degrees with forearm supinated and wrist flexion
Flex distal phalanx on flexed proximal phalanx and adducted metacarpal
Brace proximal phalanx

Force: Extend distal phalanx on proximal phalanx

Abductor Pollicis Brevis
Client supine
Elbow flexed 90 Degrees with forearm supinated
Flex/abduct radial side of proximal phalanx of thumb forward and toward base of 5th met
Maintain extension of the distal phalanx

Force: extend/adduct proximal phalanx

Flexor Pollicis Brevis
Client supine
Elbow flexed 90 Degrees with forearm supinated
Flex proximal phalanx of thumb toward 5th met head
Maintain extension of the distal phalanx

Force: Extend proximal phalanx away from 5^{th}

Adductor Pollicis Oblique Head
Client supine
Elbow flexed 90 Degrees with forearm supinated
Flex and Adduct ulnar side of thumb toward junction at the base of the 4th and 5th metacarpals
Maintain Extension of distal phalanx of the thumb.

Force: Extend proximal phalanx

Adductor Pollicis Transverse Head
Client supine
Elbow flexed 90 Degrees with forearm supinated
Flex and Adduct ulnar side of thumb toward 3rd metacarpal
Maintain extension of distal phalanx of thumb.

Force: Extend proximal phalanx

Interoseii Pollicis
Client supine
Elbow flexed 90 degrees with forearm supinated
Flex and Adduct thumb toward 2nd metacarpal
Maintain extension of the distal phalanx of the thumb.

Force: Extend proximal phalanx

Opponens Pollicis Flexor Division
Client supine
Elbow flexed 90 degrees with forearm supinated
Flex and Abduct 1st metacarpal by touching tip of thumb to 5th finger
Extend the proximal phalanx of thumb.

Force: Extend 1st metacarpal

Opponens Pollicis Abductor Division
Client supine
Elbow flexed 90 degrees with forearm supinated.
Flex and Abduct 1st metacarpal by touching tip of thumb to 5th finger
Extend the proximal phalanx of thumb.

Force: Adduct 1st metacarpal

42. Micro Order 42, Muscle Test

Extensor Digitorum Medial Division
Client supine
Elbow flexed 90 degrees with forearm pronated and slight wrist flexion**
Extend 4th and 5th digits (1 at a time)
Maintain extension at inter-phalangeal joints

Force: Flex base of proximal phalanx of 4th and 5th digits
*Grip fingers as a whole

Extensor Digitorum Lateral Division
Client supine
Elbow flexed 90 degrees with forearm supinated and wrist extended
Extend 2nd and 3rd digits (1 at a time)
Maintain extension at inter-phalangeal joints

Force: Flex base of proximal phalanx of 2nd and 3rd digits
*Grip fingers as a whole

Extensor Indicis
Client supine
Elbow flexed 90 Degrees with forearm supinated
Extend and adduct 2nd digit

Force: Flex and abduct 2nd digit

Extensor Digiti Minimi
Client supine
Elbow flexed 90 degrees with forearm supinated
Extend and adduct the 5th digit

Force: Flex and abduct the proximal phalanx of the 5th digit through sagittal plane

4th Dorsal Interossei
Client supine
Elbow flexed 90 degrees with forearm supinated
Extend and abduct 4th digit toward 5th

Force: Adduct 4th digit toward 3^{rd}

3rd Dorsal Interossei
Client supine
Elbow flexed 90 Degrees with forearm supinated
Extend and abduct 3rd digit toward 4th

FORCE: Adduct 3rd digit toward 2nd

2nd Dorsal Interossei
Client supine
Elbow flexed 90 Degrees with forearm supinated
Extend and abduct 3rd digit toward 2nd

Force: Adduct 3rd digit toward 4^{th}

1st Dorsal Interossei
Client supine
Elbow flexed 90 degrees with forearm supinated
Extend and abduct 2nd digit toward 1st

Force: Adduct 2nd digit toward 3^{rd}

43. Micro Order 43, Muscle Test

Flexor Digitorum Profundus Medial Division
Client supine
Elbow flexed 90 Degrees with forearm supinated and slight wrist extension**
Flex distal phalanx while extending proximal and distal phalanx of 4th and 5th digits

Force: Extend distal phalanx of 4th and 5th digits

Flexor Digitorum Profundus Lateral Division
Client supine
Elbow flexed 90 Degrees with forearm supinated and slight wrist extension
Flex distal phalanx while extending proximal and distal phalanx of 2nd and 3rd digits

Force: Extend distal phalanx of 2nd and 3rd digits

Flexor Digitorum Superficialis: Medial Division
Client supine
Elbow flexed 90 Degrees with forearm supinated and slight wrist extension**
Flex middle phalanx while extending distal phalanx of the 4th and 5th digits

Force: Extend middle and distal phalanx of 4th and 5th digits

Flexor Digitorum Superficialis: Lateral Division
Client supine
Elbow flexed 90 Degrees with forearm supinated and slight wrist extension
Flex middle phalanx while extending distal phalanx of the 2nd and 3rd digits

Force: Extend middle and distal phalanx of 2nd and 3rd digits

4th Lumbrical
Client Supine
Elbow flexed 90 Degrees with forearm supinated
Flex and adduct 5th digit
Brace base of proximal phalanx of 5th metacarpal while maintaining middle and distal extension
Force: Extension of the 5th digit

3rd Lumbrical
Client Supine
Elbow flexed 90 Degrees with forearm supinated
Flex 4th digit
Brace base of proximal phalanx of 4th metacarpal while maintaining middle and distal extension
Force: Extension of the 4th digit

2nd Lumbrical
Client Supine
Elbow flexed 90 Degrees with forearm supinated
Flex 3rd digit
Brace base of proximal phalanx of 3rd metacarpal while maintaining middle and distal extension
Force: Extension of the 3rd digit

1st Lumbrical
Client Supine
Elbow flexed 90 Degrees with forearm supinated
Flex 2nd digit
Brace base of proximal phalanx of 2nd metacarpal while maintaining middle and distal extension
Force: Extension of the 2nd digit

3rd Palmar Interossei
Client supine
Elbow flexed 90 Degrees with forearm supinated
Flex and adduct proximal phalanx of 5th digit toward 4th

Force: Extend and Abduct 5th digit away from 4^{th}

2nd Palmar Interossei
Client supine
Elbow flexed 90 Degrees with forearm supinated
Flex and adduct proximal phalanx of 4th digit toward 3rd

Force: Extend and Abduct 4th digit away from 3rd

1st Palmar Interossei
Client supine
Elbow flexed 90 Degrees with forearm supinated
Flex and adduct proximal phalanx of 2nd digit toward 3rd

Force: Extend and Abduct 2nd digit away from 3^{rd}

Flexor Digiti Minimi
Client supine
Elbow flexed 90 Degrees with forearm supinated
Flex proximal phalanx of the 5th digit

Force: Extend proximal phalanx of 5th digit

Abductor Digiti Minimi Flexor Division
Client supine
Elbow flexed 90 Degrees with forearm supinated
Flex and abduct the proximal phalanx of 5th digit

Force: Extend proximal phalanx of 5th digit

Abductor Digiti Minimi Abductor Division
Client supine
Elbow flexed 90 Degrees with forearm supinated
Flex and abduct proximal phalanx of 5th digit down and away from ulna

Force: Extend and adduct proximal phalanx of 5th digit

Oponens Digiti Minimi Manus Flexor Division
Client supine
Elbow flexed 90 Degrees with forearm supinated
Flex and abduct 5th metacarpal

Force: Extend 5th metacarpal

Oponens Digiti Minimi Manus Abductor Division
Client supine
Elbow flexed 90 Degrees with forearm supinated
Flex and abduct 5th metacarpal

Force: Extend and adduct 5th metacarpal

Palmaris Brevis
Client supine
Elbow flexed 90 Degrees with forearm supinated
Flex and adduct 5th metacarpal to Approximate toward 1st metacarpal

Force: Separate 5th metacarpal from 1st

### Systems and Kits

Embodiments provided herein also include systems and kits for facilitating and enhancing the maintenance of a subject's muscle set-points.

Kits in accordance with the present disclosure include exercise instructions and corresponding exercise equipment for properly stressing (exercise) a subject's muscle using the same basic macro and micro hierarchy shown above. Kits may include an instruction sheet and figures showing the exercise order for maximum benefit for any one muscle pattern or for any two or more patterns, up to instructions for all 43 movement patterns. Kits also may include instructions sheets and figures on MSAS for each muscle in the 43 movement patterns. Optionally, appropriate exercise equipment for general stress to a primary or secondary muscle is provided in a kit. Exercise equipment may include a treadmill. Exercise equipment in some aspects is designed to support ACM&S.

Systems in accordance with the present disclosure include kits and memory/computing devices for keeping track and personalizing a subject's muscle contractile profile at any one time. For example, a computer that stores and updates a subject's treatment and/or exercise status based on the embodiments herein. The memory/computing device could track muscle set points (primary and secondary) based on a health care professional's input. Objective criteria could be used for input based on the health care professional assessment and overall treatment or exercise regimes developed based on a subject's status. In addition, video of the subject's gait over a period of time may be included, where the video can be used to verify improvement to the subject's gait.

### An Illustrative Embodiment

A patient having pain or muscle dysfunction comes to the office for enhanced muscle treatment in accordance with the embodiments described herein. The patient is new to the office and has not previously been treated by the health care professional. The patient is initially stressed using MSAS for muscle pattern 1 (macro and micro, bilateral). MSAS in this case establishes the priority of pattern 1 for the new patient. MSAS on pattern 1 is followed by testing and treatment of each muscle in the pattern using AMC&S and DFAMAT. At this point the patient has had a specific stress, test, and treatment for every muscle in muscle pattern 1 (performed bilaterally). This process is repeated for every pattern up to pattern 43. At any given time, at the discretion of the practitioner, the patient is then put on an escalating speed treadmill for one minute, such that the patient is unable to keep up by the end of the minute. The health care professional notes any obvious dysfunction in the patient's gait for up to a one minute period. The patient is now tested by the AMC&S, and pattern 1 is now demonstrated to have weakness. The MSAS is then applied, the client will be treated via DFAMAT for every muscle in muscle pattern 1. Pattern 1 will be treated fully through the progressive implementation of MSAS, AMC&S and DFAMAT until the primary muscle stays strong after the MSAS (see previous embodiment). At this point the health care profession has determined that muscle pattern 1 has been sufficiently activated and treated to move onto muscle pattern 2. The health care professional determines if it beneficial to repeat the gait stress to draw out pattern 2. In this illustration, utilizing the gait stress, the health care professional works his or her way through all 43 muscle patterns, both macro and micro. The patient has completed the process and made significant strides at effectively treating the patient's dysfunctional gait.

The patient returns in one week or a reasonable time period determined by the practitioner, to continue the process. On the second visit, utilizing the gait stress, the health care professional continues to work through the macro pattern (primary muscles) for each of the 43 muscle groups, bilaterally. This process continues over a period of several months, where the patient's muscles are being tied back into the patient's CPG. In addition, because the patient is now using a functional gait, the patient's gait pattern continues to improve even in the absence of new treatment sessions.

What is claimed is:

1. A method for facilitating a subject's gait, comprising:
applying a compound stress to a first muscle in a first pattern, bilaterally, the first muscle having a highest priority in an established hierarchy of muscles within a muscle group;
determining whether the first muscle, bilaterally, is in need of treatment based on the first muscle's response to the compound stress;
administering treatment to the first muscle, bilaterally, if the fist muscle requires treatment;
moving to a second muscle, bilaterally, in the established hierarchy of muscles within the muscle group; and
continuing to apply a compound stress, testing, and treatment, for each muscle in priority;
wherein the treatments lead to an improved gait for the subject.

2. The method of claim 1 wherein the compound stress comprises a gait stressor and a specific stressor, the specific stressor being appropriate for each muscle in priority.

3. The method of claim 2 further comprising the specific stress resulting in the muscle in the muscle group being placed in a weakened state requiring treatment.

4. The method of claim 3 wherein the treatment to the weakened muscle is DFAMAT.

5. The method of claim 4 wherein the gait stress is subjecting the subject to walking on an escalating speed treadmill for thirty to ninety seconds.

6. The method of claim 5 wherein the gait stress is forty five to sixty seconds.

7. The method of claim 1 wherein the method is repeated at least once a week for six weeks, then proceed to maintenance program at a practitioners discretion.

8. A method for improving gait mechanics in a subject, comprising: reprograming the subject's Central Pattern Generator, wherein the reprogramming is accomplished by using a compound stressor in combination with testing and treatment using the muscle priority found in the subject's 43 muscle patterns.

9. The method of claim 8 wherein the compound stressor comprises at least a general stressor; and wherein the general stressor is walking on an escalating treadmill.

10. The method of claim 8 wherein the compound stressor comprises at least a specific stressor, and wherein the specific stressor is MSAS.

11. The method of claim 9 wherein the treatment is DFAMAT.

12. A kit for supporting a subject's gait comprising:
instructions for improving gait based on a hierarchical pattern for the 43 muscle groups; and
a treadmill appropriate for escalating a walking speed for the subject;
wherein use of the treadmill is provided to be coordinated with the instructions specific to improving the subject's gait.

13. The kit of claim 12 wherein the instructions includes illustrations for exercises that reinforce a non-dysfunctional gait pattern,
wherein treatment to the target muscle results in an increase in the target muscle's set point or requires an application of a biologic to the target muscle.
